(19) United States
(12) Patent Application Publication
(10) Pub. No.: US 2025/0341653 A1
(43) Pub. Date: Nov. 6, 2025

(54) ROCK CHARACTERIZATION SYSTEM

(71) Applicant: Schlumberger Technology Corporation, Sugar Land, TX (US)

(72) Inventors: Yawar Saeed, Adelaide (AU); Dong Tran, Ho Chi Minh City (VN); Bradley Martin, Kuala Lumpur (MY)

(21) Appl. No.: 19/198,194

(22) Filed: May 5, 2025

Related U.S. Application Data
(60) Provisional application No. 63/642,858, filed on May 5, 2024.

(51) Int. Cl. G01V 8/02; G01N 33/24; G06V 10/82; G10L 15/00

(52) U.S. Cl. CPC G01V 8/02; G01N 33/24; G06V 10/82; G10L 15/00

(57) ABSTRACT

A method can include receiving rock sample imagery of rock; generating characterizations of the rock based at least in part on the imagery using one or more machine learning models; and outputting the characterizations of the rock.

### RELATED APPLICATION

This application claims priority to and the benefit of a U.S. provisional application having Ser. No. 63/642,858, filed 5 May 2024, which is incorporated by reference herein in its entirety.

### BACKGROUND

A resource field may be an accumulation, pool or group of pools of one or more resources (e.g., oil, gas, oil and gas, minerals, etc.) in a subsurface environment. A bore may be drilled into an environment where the bore may be utilized to acquire samples such as, for example, core samples of rock, rock cuttings, etc. A rock sample may be in the form of a core, a section of core, collections cuttings/rock chips, fines/small particles, surface rock samples, directly on exposed rock formations, etc.

A rig may be a system of components that may be operated to form a bore in an environment, to transport equipment into and out of a bore in an environment, etc. As an example, a rig may include a system that may be used to drill a bore and to acquire information about an environment, about drilling, etc. A resource field may be an onshore field, an offshore field or an on-and offshore field. A rig may include components for performing operations onshore and/or offshore. A rig may be, for example, vessel-based, offshore platform-based, onshore, etc.

Field planning may occur over one or more phases, which may include an exploration phase that aims to identify and assess an environment (e.g., a prospect, a play, etc.), which may include drilling of one or more bores (e.g., one or more exploratory wells, etc.). Other phases may include appraisal, development and production phases. As an example, a production phase may be a mining phase.

In various instances, material from drilling operations and/or one or more other extraction operations may be assessed, for example, to characterize a formation, etc. For example, consider assessment of samples of rock of a formation. Such an assessment may depend on human observations of the samples, which may be subjective and inconsistent. Where characterizations based on samples can be improved, one or more field operations, workflows, etc., may be improved.

### SUMMARY

A method can include receiving core sample imagery of rock of rock; generating characterizations of the rock based at least in part on the imagery using one or more machine learning models; and outputting the characterizations of the rock. A system can include a processor; memory operatively coupled to the processor; a network interface; and processor-executable instructions stored in the memory to instruct the system to: receive core sample imagery of rock; generate characterizations of the rock based at least in part on the imagery using one or more machine learning models; and output the characterizations of the rock. One or more computer-readable storage media can include computer-executable instructions executable to instruct a computing system to: receive core sample imagery of rock; generate characterizations of the rock based at least in part on the imagery using one or more machine learning models; and output the characterizations of the rock. Various other apparatuses, systems, methods, etc., are also disclosed.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

Features and advantages of the described implementations may be more readily understood by reference to the following description taken in conjunction with the accompanying drawings.

FIG. 1 illustrates examples of equipment;

FIG. 2 illustrates examples of a process, a core, and a core sample;

FIG. 3 illustrates examples of cores and equipment;

FIG. 4 illustrates an example of a graphical user interface;

FIG. 5 illustrates an example of a system;

FIG. 6 illustrates examples of machine learning techniques;

FIG. 7 illustrates an example of a machine learning technique;

FIG. 8 illustrates an example of a workflow;

FIG. 9 illustrates an example of a workflow;

FIG. 10 illustrates an example of a workflow;

FIG. 11 illustrates an example of a workflow;

FIG. 12 illustrates an example of a workflow;

FIG. 13 illustrates an example of a workflow;

FIG. 14 illustrates examples of workflows;

FIG. 15 illustrates examples of graphical user interfaces;

FIG. 16 illustrates an example of a graphical user interface and examples of equipment;

FIG. 17 illustrates examples of graphical user interfaces;

FIG. 18 illustrates an example of a method and an example of a system; and

FIG. 19 illustrates an example of computing system.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims.

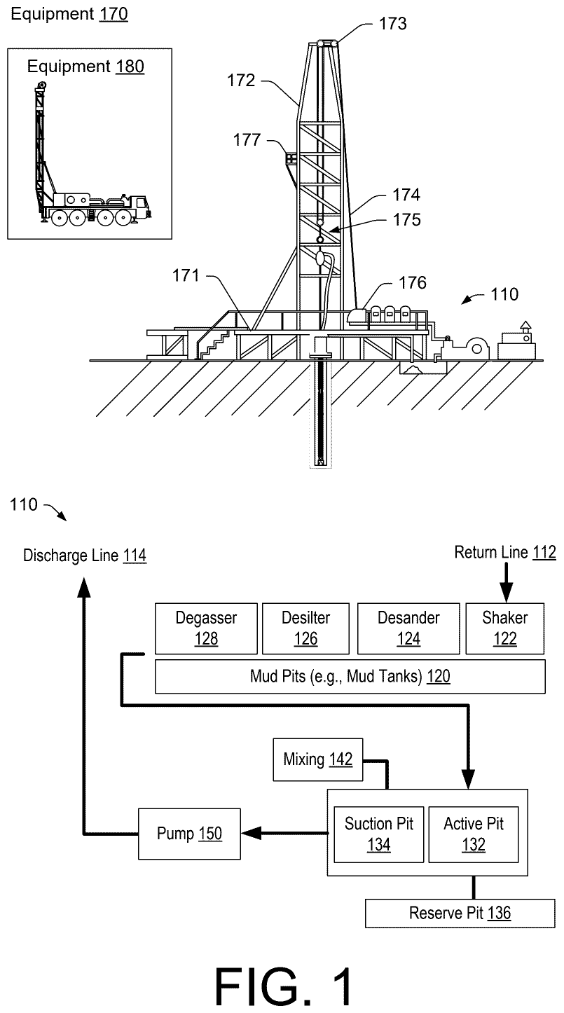

FIG. 1 also shows an example of equipment 170 and an example of equipment 180. Such equipment, which may be systems of components, may be suitable for use in a geologic environment. While the equipment 170 and 180 are illustrated as land-based, various components may be suitable for use in an offshore system.

The equipment 170 includes a platform 171, a derrick 172, a crown block 173, a line 174, a traveling block assembly 175, drawworks 176 and a landing 177 (e.g., a monkeyboard). As an example, the line 174 may be controlled at least in part via the drawworks 176 such that the traveling block assembly 175 travels in a vertical direction with respect to the platform 171. For example, by drawing the line 174 in, the drawworks 176 may cause the line 174 to run through the crown block 173 and lift the traveling block assembly 175 skyward away from the platform 171; whereas, by allowing the line 174 out, the drawworks 176 may cause the line 174 to run through the crown block 173 and lower the traveling block assembly 175 toward the platform 171. Where the traveling block assembly 175 carries pipe (e.g., casing, etc.), tracking of movement of the traveling block 175 may provide an indication as to how much pipe has been deployed.

A derrick may be a structure used to support a crown block and a traveling block operatively coupled to the crown block at least in part via line. A derrick may be pyramidal in shape and offer a suitable strength-to-weight ratio. A derrick may be movable as a unit or in a piece-by-piece manner (e.g., to be assembled and disassembled).

As an example, drawworks may include a spool, brakes, a power source and assorted auxiliary devices. Drawworks may controllably reel out and reel in line. Line may be reeled over a crown block and coupled to a traveling block to gain mechanical advantage in a “block and tackle” or “pulley” fashion. Reeling out and in of line may cause a traveling block (e.g., and whatever may be hanging underneath it), to be lowered into or raised out of a bore. Reeling out of line may be powered by gravity and reeling in by a motor, an engine, etc. (e.g., an electric motor, a diesel engine, etc.).

As an example, a crown block may include a set of pulleys (e.g., sheaves) that may be located at or near a top of a derrick or a mast, over which line is threaded. A traveling block may include a set of sheaves that may be moved up and down in a derrick or a mast via line threaded in the set of sheaves of the traveling block and in the set of sheaves of a crown block. A crown block, a traveling block and a line may form a pulley system of a derrick or a mast, which may enable handling of heavy loads (e.g., drillstring, pipe, casing, liners, etc.) to be lifted out of or lowered into a bore. As an example, line may be about a centimeter to about five centimeters in diameter as, for example, steel cable. Through use of a set of sheaves, such line may carry loads heavier than the line could support as a single strand.

As an example, a derrickman may be a rig crew member that works on a platform attached to a derrick or a mast. A derrick may include a landing on which a derrickman may stand. As an example, such a landing may be about 10 meters or more above a rig floor. In an operation referred to as trip out of the hole (TOH), a derrickman may wear a safety harness that enables leaning out from the work landing (e.g., monkeyboard) to reach pipe in located at or near the center of a derrick or a mast and to throw a line around the pipe and pull it back into its storage location (e.g., fingerboards), for example, until it a time at which it may be desirable to run the pipe back into the bore. As an example, a rig may include automated pipe-handling equipment such that the derrickman controls the machinery rather than physically handling the pipe.

As an example, a trip may refer to the act of pulling equipment from a bore and/or placing equipment in a bore. As an example, equipment may include a drillstring that may be pulled out of a hole and/or placed or replaced in a hole. As an example, a pipe trip may be performed where a drill bit has dulled or has otherwise ceased to drill efficiently and is to be replaced. In various instances, depending on borehole conditions, a risk of pipe getting stuck may lead to various issues, which can include catastrophic failure. For example, if a portion of a drillstring gets stuck, it may breakoff and result in a fishing trip, drill-out, abandonment, etc. In various instances, risk of sticking may depend on hole cleaning, as may be expected to occur with adequate mud flow (e.g., flow of drilling fluid to remove cuttings, etc.).

FIG. 1 also shows an example of a drilling fluid system 110. During drilling, a drilling fluid (e.g., mud) may be utilized to lubricate a drill bit, remove heat energy, carry cuttings to surface, provide for telemetry (e.g., mud-pulse telemetry), etc. For example, the drilling fluid system 110 may aim to provide for various operations, which may include one or more of removing cuttings from a well, controlling formation pressures, suspending and releasing cutting, sealing permeable formations, maintaining wellbore stability, minimizing formation damage, cooling, lubricating and supporting a bit and drilling assembly, transmitting hydraulic energy to one or more downhole tools and/or a bit, ensuring adequate formation evaluation, controlling corrosion, facilitating cementing and completion, preventing gas hydrate formation, and minimizing impact on the environment.

As shown in the example of FIG. 1, the system 110 can include a return line 112 and a discharge line 114. In the example of FIG. 1, the system 110 may include a shaker 122, a desander 124, a desilter 126, and a degasser 128 associated with various mud pits 120 (e.g., mud tanks) that can receive drilling fluid via the return line 110 and output processed drilling fluid to an active pit 132 that may be in fluid communication with a suction pit 134 and a reserve pit 136 where the suction pit 134 may be in fluid communication with a pump 150 that can pump drilling fluid to the discharge line 114. As an example, one or more mixing units 142 may be included, for example, for addition of one or more materials to the drilling fluid before it is pumped to the discharge line 114.

As an example, the system 110 may be utilized for one or more types of operations, which may include drilling, wireline, completions, blow out control, etc. As to completions, as an example, a cementing operation may include pumping and/or receiving of drilling fluid where cement may be positioned between casing and a borehole wall.

As an example, cuttings may be retrieved at surface, for example, using one or more of the components of the system 110. Cuttings can be produced as rock is broken by a drill bit advancing through a subsurface environment. Cuttings may be carried to surface by drilling fluid (e.g., mud) circulating from one or more openings of a tool string such as, for example, openings of a drill bit of a drillstring. Drill cuttings may be separated from fluid using one or more types of equipment such as, for example, shale shakers, centrifuges, cyclone separators, etc. In cable-tool drilling, cuttings may be periodically bailed out of a bottom of a borehole. In auger drilling, cuttings may be carried to surface on auger flights.

In various instances, cuttings may be analyzed to provide information as to a borehole being drilled, formations being drilled into, drilling efficiency, bit condition (e.g., bit wear, etc.), risk of stuck pipe (e.g., sticking of a drillstring in a borehole due to cuttings, borehole condition, etc.), etc. While cuttings may be transported to surface via drilling fluid (e.g., mud), other material from a formation may also be transported, whether fluid (e.g., liquid or gas), solid, etc. In various instances, material from drilling equipment (e.g., a drill bit, downhole tool, etc.) may be transported to surface via drilling fluid, for example, in the instance that a component breaks, loses a part, etc. For example, a chip of a drill bit may be transported to surface via drilling fluid. As explained, material within drilling fluid as transported to surface may provide insight as to one or more types of phenomena, which may be related to one or more of operational, equipment condition, formation characteristics, etc.

As an example, equipment may be utilized to acquire a core sample, which may be referred to simply as a core. A process to acquire a core may be referred to as coring. In various instances, a core may be assessed as to characteristics germane to production of hydrocarbons. For example, consider assessing a core as to porosity, permeability, water saturation, hydrocarbon saturation, etc. In various instances, a core may be assessed as to characteristics germane to mining. For example, consider assessing a core as to a particular mineral or minerals. In various instances, a core may be assessed as to stratigraphy, for example, to understand how a subsurface region is layered. In various instances, a core may be assessed as to lithology, which may be defined as the macroscopic nature of mineral content, grain size, texture and color of rocks.

As to some examples of coring, consider rotary coring, which may utilize a rotary coring bit disposed at the end of drillpipe where rotation of the rotary coring bit cuts into material such that a core sample is captured within a bore of the drillpipe. In such an example, upon tripping the drillpipe out of the drilled bore, the core may be removed for analysis. For example, the core may be removed in sections and placed into a tray for inspection. Some types of rotary coring may include rig-based coring, which may include drillpipe coring, wireline coring, diamond coring, etc. As an example, coring may include side-wall coring. Side-wall coring may acquire a core in a borewall of a borehole that has been drilled.

As to equipment for coring, such equipment may include drillpipe with a core liner that may be disposed in a nonrotating inner barrel where a core bit is mounted to an end of an assembly where core catchers may help to retain a core within the core liner as the core bit cuts into rock to form the core. As to a wireline approach, equipment may include coring components that may be disposed into a bore of drillpipe to be descended via wireline to an end of the drillpipe for deployment and core cutting and capture.

A core sample may be a cylindrical sample of rock acquired using a core bit in conjunction with a core barrel and core catcher. In such an example, the core bit includes cutting structures (e.g., polycrystalline diamond compact (PDC), natural diamond, etc.) about a central hole. The core bit can be rotated to drill around a central cylinder of rock, which is taken in through the central hole of the bit and into the core barrel. The core barrel serves as a storage chamber for holding the rock core while a core catcher can serve to grip the bottom of the rock core and, as tension is applied to a drill string, the rock under the rock core can be broken away from an undrilled portion of formation below the rock core. The core catcher may also act to retain a core, for example, so that it does not fall out the bottom of the drill string.

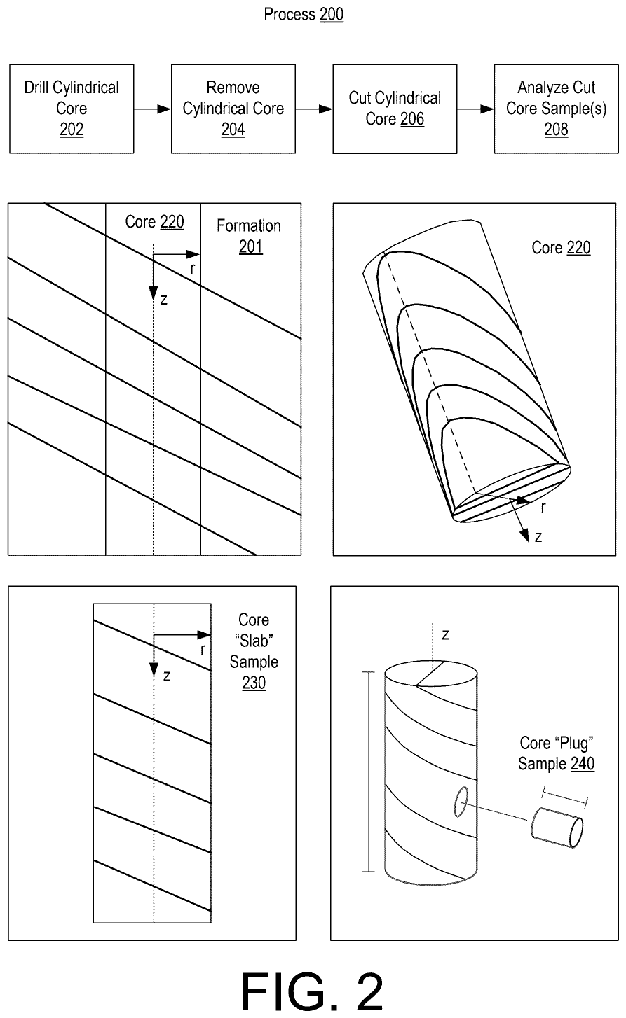

FIG. 2 shows an example of a process 200 along with graphical representations of a core 220 from a subsurface formation 201. The process includes cutting a cylindrical core 202; removing the cylindrical core 204; cutting (e.g., “slabbing” or “plugging”) the cylindrical core 206, for example, to provide a slab sample (see, e.g., the slab sample 230) and/or one or more plug samples (see, e.g., the plug sample 240); and analyzing the one or more samples 208. As illustrated, a cut parallel to the longitudinal axis can expose a planar surface of the core 220 to provide the slab sample 230, which may then be analyzed (e.g., as to layers, lithology, etc.). As illustrated, a cut into a surface of the core 220 can provide the plug sample 240, which may be within a layer to provide an analysis of a particular layer. As an example, where a core includes a large inclination of the bedding plane (e.g., dip), the core plug may be taken in a direction parallel to the bedding plane (e.g., to estimate horizontal and vertical permeability). While slab type and plug type samples are illustrated in FIG. 2, samples may be taken in one or more other angles in relation to a bedding plane (e.g., parallel, perpendicular, 45 degrees, etc.) or manners (e.g., by cutting, etc.). As an example, a core may be utilized to assess dip, where, for example, an indication as to direction of the core may be known and/or estimated. As an example, borehole imagery may be utilized where, for example, a camera may be disposed in a borehole to capture imagery of a borehole wall, which may correspond to one or more cores extracted from a subsurface region that, upon extraction, form the borehole (e.g., noting that some material may be lost due to a kerf formed by a cutting bit (e.g., consider a borehole having a diameter that is larger than that of a core where an annulus is lost due to operation of a cutting bit).

As an example, a core sample may be analyzed to determine petrophysical data. For example, analyses of a core sample may provide measurements of porosity, grain density, horizontal permeability, fluid saturation, etc. As an example, a lithologic description may be made as to one or more portions of the core sample. As an example, analyses may provide for a core gamma log and measurements of vertical permeability. As an example, measurements may be made at various pressure-temperature conditions, including room and/or formation temperature, atmospheric and/or formation confining pressure, etc. Analyses may include routine core analysis (RCA) and/or special core analysis (SCAL). As an example, one or more core analyses may be performed as described in the American Petroleum Institute (API) document “Recommended Practices for Core Analysis” (API RP 40).

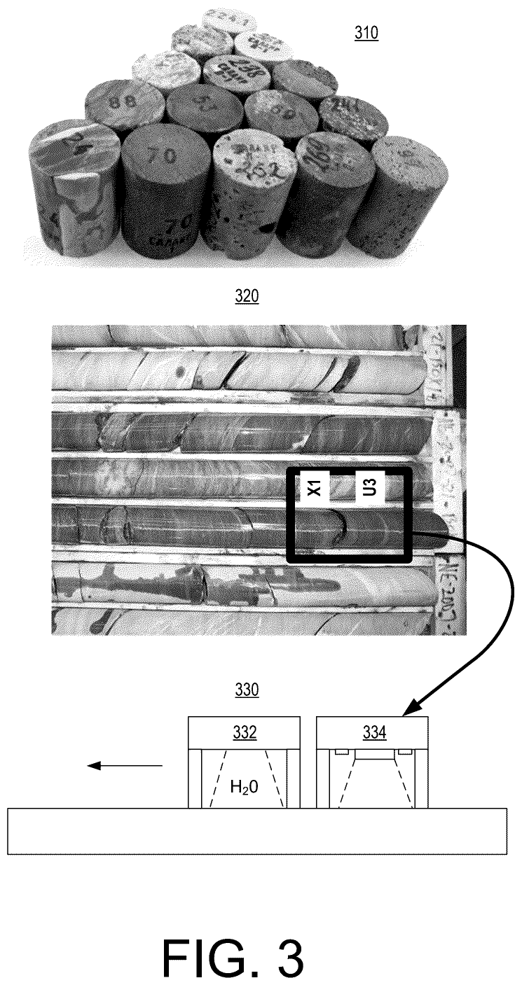

FIG. 3 shows an example of cut cores 310 (e.g., a series of cylinders), cores in a rack 320, and a system 330 that can provide for imaging cores and, for example, wetting cores. As shown, the system 330 may include a wetting unit 332 and an imaging unit 334. In such an example, the wetting unit 332 may spray water to wet a core or cores and the imaging unit 334 may capture imagery of a core or cores. Such units may be movable automatically, semi-automatically or manually. As an example, imagery may be captured where augmented reality may be utilized to provide for viewing one or more labels associated with one or more cores, portions of a core, portions of cores, etc. As an example, the imaging unit 334 may include a digital image sensor and one or more lights that may emit one or more wavelengths of light (e.g., IR, UV, VIS, etc.). As an example, the imaging unit 334 may include a lens or lenses, which may be fixed in focus and/or focusable where, for example, one or more aperture mechanisms may be included, which may provide for depth of field selection and/or adjustment. As an example, the imaging unit 334 may provide for utilization of a mobile imaging device, which may be a mobile device such as, for example, a mobile phone, a tablet, etc., which includes one or more digital image sensors, one or more lenses, etc. As an example, the imaging unit 334 may include one or more processors and memory accessible thereto, along with one or more interfaces (e.g., BLUETOOTH, WIFI, satellite, etc.). As an example, the imaging unit 334 may provide for execution of an application, which may be local and/or remote (e.g., server-based, cloud platform-based, etc.).

As an example, a system may include one or more mobile devices (e.g., phones, tablets, etc.), one or more edge devices, one or more Internet-of-Things (IoT) devices, one or more fixed CCTV devices, one or more machine vision sensors, one or more drones and/or robots, etc. As an example, imagery may include still images, frames from a video recording, frames from a streaming video, etc.

As an example, one or more of the components of the system 330 may be utilized for acquisition of imagery, which may be for core imagery, cuttings imagery, etc. As an example, as to cuttings, consider a camera directed at a shaker or shakers (see, e.g., the shaker 122 of FIG. 1) where imagery captured by the camera may be utilized to characterize cuttings as they are collected from mud.

As an example, imagery may be acquired for one or more processes that involve rocks. For example, consider one or more cameras focused on a conveyor belt of a mineral handling and/or processing facility.

As an example, field imagery may be acquired using one or more cameras as may be transported using a drone, whether air, water, and/or ground. For example, consider a robot that may be directed to investigate one or more regions to capture imagery of rocks. In such an example, the robot may be directed according to a pre-planned program (e.g., map grid, etc.), a human controlled manner, or an intelligent manner whereby direction, speed, etc., of the robot may be controlled based at least in part on one or more features of acquired imagery. In such an example, the robot may provide for local processing and/or remote processing. For example, consider an embedded machine learning-based framework and/or a remote cloud platform-based framework that may receive acquired imagery, assess such imagery, and then transmit instructions to a robot for directing the robot. While a single robot is mentioned, as an example, a fleet of robots may be utilized where imagery may be collectively assessed, for example, to control one or more members of the fleet, which may provide for effective assessment of a region in an intelligent manner.

As an example, a robot or other device may include one or more cameras that may be fixed or controllable to focus in one or more directions. For example, consider a ground facing camera and a forward-facing camera where imagery from both cameras may be utilized to intelligently control movement of a robot or other device. As an example, a robot may be an inspection “dog” that may roll, crawl, walk, etc., in remote locations and/or may be an airborne drone that may provide for capturing rock types from exposed rock formations, etc.

As an example, imagery captured by an imaging unit or imaging units may be assessed to determine one or more characteristics of a sample or samples (e.g., ground, core, cuttings, etc.). As an example, an imaging unit may provide for capture of one or more types of data, which may provide for enhancing determinations. For example, consider voice capture via a microphone, input of one or more menu selections via a graphical user interface (GUI), etc. As an example, a touchscreen display may be included where a user may interact with an imaging unit and, for example, an application (e.g., an app) via contact with the touchscreen display. In such an example, a user may touch a portion of a rendered image of a sample to identify a region for assessment where, in response, an application determines one or more characteristics of the sample.

As explained, a sample may be wetted with water, noting that one or more other fluids may be utilized. As an example, rock characteristics may provide for insights into properties of a reservoir, lithology of one or more formations, permeability of rock, indicia of one or more of traces of oil, water, and natural gas. As an example, a rock characteristic may include fossils, organics matter, etc.

As an example, a wettability assessment may provide for characterization of a preference of a surface to be in contact with one fluid rather than another. In rock, a surface may be composed of mineral grains where there may be fluid in pore space (e.g., liquid, gas, etc.). In various instances, a balance of forces (e.g., surface tensions) may control wettability. Surface tension results from the natural tendency of molecules at a fluid interface to be at a higher energy state than those in the bulk of a fluid. This tendency creates a reduced concentration of molecules close to a fluid interface, and fluid molecules pulled toward a fluid interior, imparting an adhesive force. This force, found at surfaces between immiscible fluids, is surface tension at gas/liquid boundaries and interfacial tension in liquid/liquid boundaries.

As an example, the system 330 may be utilized for core assessments for one or more purposes, which may include purposes related to mining, aquifers, oil, gas, CCUS, etc.

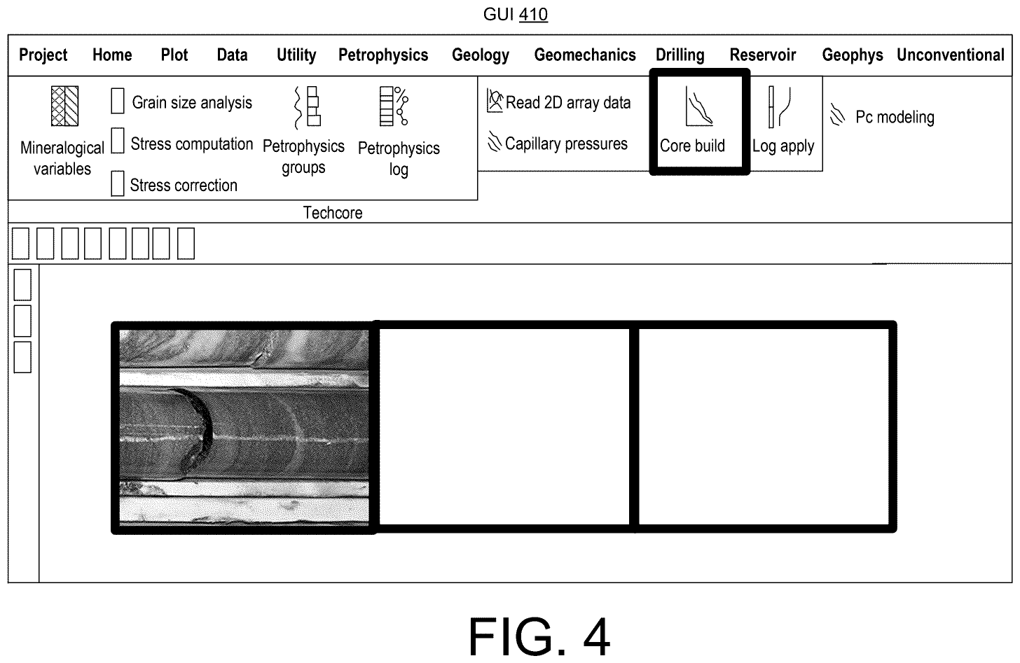

FIG. 4 shows an example of a graphical user interface (GUI) 410 that may be part of a framework executed using one or more processors, memory accessibly the at least one of the one or more processors, etc. As an example, the GUI 410 may be part of a framework such as the TECHLOG framework (SLB, Houston, Texas). As an example, the GUI 410 may be used to implement a workflow that includes analyzing cores or other rock samples. For example, a user may navigate the GUI 410 to select a core build application component, which may cause rendering of menu options that may provide for capture and/or assessment of imagery, optionally along with other data.

As shown in FIG. 4, the GUI 410 may include various options associated with sample analysis, which, in turn, may aid in characterizing rock such as reservoir rock (e.g., hydrocarbon reservoir rock, aquifer reservoir rock, carbon sequestration reservoir rock, etc.), mineral rock for mining, etc. As an example, a workflow may include one or more worksteps associated with one or more graphical controls of the GUI 410. As an example, a workflow may include one or more worksteps associated with the process 200 of FIG. 2. As an example, a workflow may include performing one or more field operations, for example, in a field from which a rock sample was extracted. As an example, a field operation may include acquiring one or more samples, drilling, injecting fluid, producing fluid, etc. As an example, a field operation may depend in part on results of an analysis of a sample of rock or samples of rock.

As explained, cores or core pieces may be placed in a rack (e.g., a tray). In various instances, a tray may be prepared by a human. As to photography, a tray may be placed in front of a camera lens where photo acquisition may be performed with visible white light, IR illumination, UV illumination, etc., (e.g., sunlight, lamps, LEDs, mirrors, lenses, etc.). As an example, photography may be performed in the field at a field site where digital imagery, whether raw and/or processed, may be transmitted to one or more destinations that may be remote from the field site. As an example, digital imagery may be compressed using one or more compression techniques (e.g., lossless, lossy, etc.), which may facilitate transmission, particularly where transmission may be via a satellite network (e.g., consider remote locations, offshore rigs, etc.), which may have a low and/or costly bandwidth. As an example, a geologist may be present at a location, whether local and/or remote for purposes of analysis. As an example, analysis may aim to extract geologically meaningful information from core imagery. As an example, a system may be a multi-spectroscopy imaging system for rock characterization. As an example, a workflow may include performing one or more actions for calibration of a multi-spectroscopy imaging system for rock characterization.

As an example, a system may be an advanced imaging system that includes components for automating one or more aspects of a rock characterization workflow for applications such as environmental applications, industrial applications, etc. As an example, such a system may include multiple emissions sources for implementation of multi-spectroscopy techniques. For example, consider dual spectroscopy techniques that can include white-light direct absorption for color and texture analysis, and UV-induced fluorescence for hydrocarbon identification; noting that UV-induced fluorescence may also provide for characterization of one or more types of minerals.

As an example, a mono-spectroscopy system and/or a multi-spectroscopy system may be applied to analysis of cores and/or one or more other types of samples. For example, consider cuttings analysis, water-hydrocarbon fluid analysis, drilling fluid analysis, etc.

As an example, a system can include a digital scope including a machine vision lens attached to a high-resolution machine vision camera, connected to a portable computing device for purposes of image calibration and acquisition. As an example, a lighting unit may provide for selection of light temperature, light color, etc.

As an example, a system may implement one or more machine learning models (e.g., ML models) for rock lithology prediction. As an example, such a system may provide for post-processing and/or real-time processing. As explained, results from a system may be rendered to a display, optionally in a manner that may utilized augmented reality such that an overlay can be rendered on an image where, for example, user interaction may occur to improve, confirm, etc., one or more rock lithology predictions. As an example, an improvement may occur in response to accessing information from an offset borehole, for example, to register depths, formation tops, rock characteristics, etc.

In mining exploration, as in oil and/or gas exploration, test boreholes may be drilled to understand subsurface rock structures and geological formations. Unlike most oil and/or gas explorations, seismic surveys tend to be rare in mining exploration; rather, a mining company may drill hundreds to thousands of shallow boreholes in an area of interest where core samples may be recovered from a number of the boreholes. In such a process, an onsite field geologist may examine cores and determine lithology (rock type) through visual observation and occasionally performance of one or more of physical tests and/or chemical tests.

In general, core imagery and core analysis data are proprietary, to be secured and confidential. For example, an irreputable entity that performs core analysis may not inform its client and, for example, utilize valuable information to acquire adjacent land and/or rights thereto. Further, reliance solely on a field geologist may result in variability from site to site and/or from field geologist to field geologist. Through a machine vision approach, core analyses may be more objective and reproducible, which may provide for using results thereof for assessment of other cores. For various reasons, an approach that is more objective, reproducible, and secure may be an improvement upon a field geologist-based approach. For example, as to security, a machine vision approach may utilize specialized coding such that individuals with a need to know can access the specialized coding. For example, X1 may represent a particular mineral where rendering of a label X1 to an image on a display lacks meaning to an individual that does not have access to the code. Further, codes may be updated on a determined basis to help protect core analysis results.

As an example, a rock characterization system may provide for sample analysis where results may be output along with confidence (e.g., probability, uncertainty, etc.). In such an approach, lithology from a field site may be assessed as to accuracy in an expeditious manner, which may provide for reduction of waste (e.g., in a LEAN sense) for one or more following workflows. For example, if a field geologist believes that a field observation is accurate where, in actuality, it is later found to be incorrect (e.g., weeks, months or years later), such an error can have a profound impact on one or more resulting workflows. In such an example, consider an error not being uncovered until one or more core samples are analyzed in a lab or when geophysical numerical are models are built. The impact of a mistake can mean time and cost to correct and rebuild models, or cause incorrect decision to be made.

As an example, a rock characterization system may provide for expeditious results from at least imagery captured at a field site. Such a system may help to ascertain an initial rock characterization along with information that indicates whether or not the initial rock characterization is reliable or not. In such an approach, if the system indicates that the initial rock characterization is not reliable, one or more workflows may be delayed until in-lab analyses may be performed (e.g., by physically transporting core samples to a lab, etc.); whereas, if the initial rock characterization is deemed sufficiently reliable, then one or more workflows may be planned and/or executed using the initial rock characterization and, depending on timing, one or more follow-up characterizations, if performed.

As to reasons for mis-classification, there can be many. For example, certain rock types appear visually to be very similar, they have the same color but may only vary in grain size, for other physical or chemical properties, for example. As an example, rocks may even be from different families (e.g., Metamorphic, Igneous or Sedimentary) yet appear, to the human eye, to be the same.

Further compounding challenges faced field geologist assessments can be locations of field sites and/or conditions thereof. For example, exploration areas may be extremely remote with limited facilities/resources; observations may be made outside when it may be very hot and/or in direct sunlight; personnel may be working long shifts under tough conditions; etc. A geologist's experience and experience with certain rock types may be additional factors.

As an example, a rock characterization system may implement one or more AI/ML techniques to determine rock type, which may help to reduce the number of misclassification errors made in the field.

As an example, lithology prediction can address the challenge of efficiently and accurately classifying lithology in geological samples (e.g., rock samples). As explained, a rock characterization system may utilize and/or include one or more mobile apps, cloud storage, transmission protocols, communication channels, a web app, one or more ML models, etc.

As an example, a mobile app or a number or suite of apps may be installed on a mobile device that may be used by a geologist or other person at a field location, remote field location or other location. In such an example, the app may allow an individual to identify lithology of a rock sample, etc. by taking capturing imagery (e.g., still, video, etc.) of a rock sample where one or more ML models may be built into the app and/or operatively coupled thereto to predict rock type. As an example, detailed information about rock type may be provided as a reference to a geologist. As an example, a further testing/evaluation process may be provided to guide a geologist through one or more physical tests and/or chemical tests to determine rock type. For example, to reduce uncertainty, a rock characterization system may generate one or more recommendations that may help to provide for improved assessment. In such an approach, the system may utilize one or more approaches to arrive at a definitive test and/or a flowchart (e.g., decision tree) as to one or more tests to provide for an improved determination, which may be based on available resources (e.g., distance to lab, availability of testing in the field, etc.). As an example, a system may “know” when a scenario is present as to one or more of certain rock lithologies where physical appearance may be the same, but the physical and/or chemical properties are different. As an example, a workflow may recommend or call for testing for physical and/or chemical properties that may help differentiate between lithologies. For example, for certain lithologies, a framework (e.g., a framework app, etc.) may provide for proposing one or more tests and, for example, allowing a user and/or a machine to enter relevant results. As an example, where machine learning is utilized, such results may be feedback that may provide for training, re-training, etc., to improve performance of a framework, a system, an imaging system, a robot, etc.

As an example, a system may provide for entry of input, which may be via menu selection, interactions with a GUI, entry of data, metadata, etc. For example, consider entry of other data about a sample such as well name and location, rock sample depth and/or other observations about a sample. As an example, observations/notes and other events may be typed or captured by a voice to text module that generates written notes dictated into a mobile device by an individual (e.g., a geologist or other individual). As an example, if desired for more accurate identification, results of one or more specific physical and/or chemical measurements may also be collected. As an example, measurement results may be typed into a mobile device and/or recorded via one or more types of communication channels (e.g., consider wireless enabled measurement devices for example a hardness tester with BLUETOOTH, WIFI, IR, etc.).

As an example, imagery, data and results may be stored in an app on a mobile device. As an example, where a device is connected to a mobile, a cellular, a satellite, a WIFI, etc. network, data may be uploaded and stored in storage of a cloud platform where results may be accessible via a web app. As an example, once uploaded to the cloud, data may be used in one or more types of frameworks (e.g., TECHLOG, etc.). As an example, where connectivity does not exist, data may be uploaded at a later time when connectivity does exist. In various instances, data may not be uploaded to a cloud server, for example, if long term storage is not desired. As explained, one or more techniques may be employed as to data security. For example, consider encryption of data, encryption of imagery, compression combined with encryption, etc.

As an individual uses an app to predict lithology of one or more samples from a well, as an example, the app may generate a profile and/or image of the lithology of a length of the well (see, e.g., the example of FIG. 4). As an example, an app may provide for comparing a portion of rock characterizations of one well to one or more other wells, which may be in the same field/region and/or which may be deemed to be one or more analogs. As an example, an app may provide for one or more of offset borehole comparison and/or analog comparison. In such an approach, one or more types of search, matching, etc., techniques may be applied. For example, consider a search based on distances between formation tops, interfaces, types of rock, etc.

As an example, a system may provide for rendering of a 3D view of a field/area, which may be generated to better understand how layers of formations/rocks may vary. Such an approach may also allow current results to be checked against one or more other borehole, formations, etc. As an example, a system may provide for corrections, adjustments, etc., to be performed manually, semi-automatically and/or automatically as to rock lithology. As explained, a system may aim to provide output at a field site that may include one or more metrics indicative of confidence (e.g., reliability of a prediction or predictions). As an example, a GUI may be rendered to a display with imagery of a core or cores where an overlay or other indicators may provide for confidence along the core or cores. In such an approach, portions of a core or cores with higher confidence may be utilized to perform one or more comparisons, searches, etc., where results therefrom may be utilized to improve confidence of one or more portions with lower confidence. In such an example, a length of a core may be filled in based on one or more confidence assessments, which may be output from one or more ML models. As an example, an automated approach may be tiered where, for example, output from one process is utilized by one or more subsequent processes to improve overall results.

As an example, a system may provide for loading of images previously captured, for example, consider loading to a cloud platform server where lithology may be predicted. As an example, an edge framework may be implemented at a field site that may provide sufficient computational resources for local processing. In such an example, the edge framework may include a network interface suitable for transmission of imagery (e.g., raw, processed, etc.) and receipt of assessment results. remote processing via a network interface. As an example, an edge framework may provide for communication with one or more imaging devices (e.g., cameras, etc.).

As explained, one or more apps may provide for analyzing single images and/or video. As an example, video may be captured and analyzed by treating each frame of the video as an image. As an example, imagery may include overlap, which may help to provide for registration of one image to another image. As an example, for purposes of input to a ML model or ML models, output from one image may be utilized as input to another image. In such an approach, generation of output may be kickstarted by overlap. For example, 10 percent overlap may be utilized such that portions of images may be assess multiple times. In such an approach, where a second assessment provides for greater confidence, the first assessment may be overwritten. For example, consider a first 5 cm length where the 0.5 cm is overlapped with 0.5 cm of another, second 5 cm length. In such an example, the output from the 0.5 cm of the first 5 cm length may be utilized as input for the first 0.5 cm of the second 5 cm length. In such an approach, if the assessment of the second 5 cm length, as to the first 0.5 cm is with higher confidence than the utilized input, then the first 5 cm length may be updated as to its last 0.5 cm. In such an approach, a portion of a prior length may be utilized as an initial guess as to a portion of a subsequent length.

As an example, a system may utilize a Convolutional Neural Network (CNN) model trained using transfer learning and dual transfer learning techniques, enabling it to effectively classify core images by rock lithology. As an example, images captured with a mobile app and/or uploaded to a website may be resized, cropped and other filters/processes applied. As an example, an image may be passed into a trained ML model that generates predicted rock lithology and associated probability outputs. As explained, probability output(s) may provide an indication of confidence.

As an example, in some cases, it may be desirable to first determine a rock family with a rock family model and then use a specific model trained for the specific family to determine. In such an example, models may be trained for rock family and specific lithology models trained for each rock family.

As an example, a training workflow may involve iterative model improvements for improved lithology classification accuracy. As an example, a base model may be trained with images from one or more sources such as, for example, a core library, a cuttings library, etc. As an example, a base model may be improved by then training it with images captured in the field, for example, with the same or similar conditions to the end users.

As an example, an automated process may be implemented by a system to extract images and related data from a library (e.g., a database, a collection of core images, a collection of geological reports, etc.) where labeling of images may be based on expert geologist classifications. As an example, images may be resized, cropped sorted by lithology type. Such a process may be applied to images and/or reports from one or more sources such as national databases, lab databases, customer databases, etc. As to reports, they may be saved as images, PDF documents, etc. As an example, a framework may provide for digitization, optical character recognition (OCR), object recognition, etc. As an example, a framework may provide for extraction of imagery and text where, for example, one or more machine learning models may be utilized to assess the imagery and/or text, individually or in combination. As an example, a large language model (LLM) may be utilized for text assessments, which may include generative text assessments. As an example, an LLM may be provided that can link imagery and text for purposes of assessments, associations, etc.

As an example, a rock characterization system may provide for a fast, accurate, and scalable solution for lithology classification, along with one or more other tools that may be helpful for a field geologist and/or another individual in the field.

As an example, a system may provide various digital tools, which may be on an on-demand basis, which may depend on type of connectivity available to a device or devices in the field. In such an example, an individual (e.g., field geologist, etc.) may capture data digitally at a remote field location and store these data in a cloud database for traceability and connection to one or more appropriate petrotechnical frameworks, which may provide for further modelling and/or analysis. As an example, a system may be implemented in part by use of a mobile device such as a phone or tablet with sufficient processing capabilities (e.g., one or more CPUs, one or more GPUs, etc.), which may be operable with and/or without connectivity to a network or networks. As an example, an app may be executed on a mobile device in the field, which may be at a remote location. As an example, a workflow may be formulated to be intuitive with minimal training demand. As explained, a workflow may be facilitated through use of one or more imaging techniques, rendering techniques, etc. As explained, augmented reality may be implemented to help guide a user in a rock characterization workflow as to one or more portions of a core.

As explained, a system may provide an emphasis on field validation and integration of field images, for example, through use of a dual transfer learning approach. As an example, a system may provide for robust field implementation at least for local capture of imagery and local app execution, which may depend on one or more types of relatively lightweight machine learning frameworks (e.g., consider Tensor Flow Lite (TFL), Google, Mountain View, California). As an example, versions of ML models may be generated remotely and provide for local updating where connectivity is available and/or where media may be transported physically to a site (e.g., via ground vehicle, water vehicle, air vehicle, etc.). As to robustness, an app may provide for handling of various conditions, which may be, for example, built into one or more ML modelling approaches. For example, consider an approach that may automatically gather information as to local conditions under which imagery have been captured, which may include, for example, conditions such as lighting (e.g., sunlight and/or artificial light), temperature, humidity, surface wetting, etc. In such an approach, an app may automatically provide for handling of condition effects as to texture, color, etc. By incorporating field validation and leveraging dual transfer learning, an app may provide for achieving high accuracy in predicting rock sample lithology using mobile imagery captured in diverse environmental conditions. Such an approach can help to ensure that geologists can rely on results, for example, as to precise lithology classification in the field, ultimately enhancing efficiency and accuracy in geological analysis. As explained, confidence may be output where, to improve confidence, one or more suggestions may be generated, which may, for example, depend on availability of equipment to perform one or more types of tests, re-capture imagery under one or more different conditions, etc. As an example, where confidence may be due to lighting, surface condition, focus, movement, etc., a recommendation may be to re-capture imagery of a portion of a rock sample. For example, consider a scenario where a cloud may pass by the sun such that a portion of a core may be exposed less than another portion. In such an example, an app may instruct an individual to wait until a cloud passes or until lighting is more even for a number of captured images of one or more portions of a core.

As an example, a rock characterization system may implement one or more AI/ML techniques for improved geology assessments, for example, to improve lithology classification processes in the field at field sites. For example, by combining artificial intelligence (AI) with image recognition capabilities, a system may empower individuals (e.g., geologists, etc.) to analyze core samples swiftly and accurately in various geological settings. For example, consider utilization of one or more LLMs, one or more vision language models (VLMs), etc.

As an example, a VLM may include an architecture that includes a vision encoder, a projector, and an LLM. For example, consider a vision encoder that may include components of a CLIP-based model with a transformer architecture that has been trained on image-text pairs, giving the vision encoder an ability to associate images and text. As an example, a projector may be structured as a set of layers that translate output of a vision encoder into a form suitable for use as input to an LLM, for example, interpreted as image tokens. As an example, a projector may be a line layer like Large Language-and-Vision Assistant (LLaVA) and VILA, or more complex like cross-attention layers used in Llama 3.2 Vision (Meta Platforms, Menlo Park, California).

As explained, a system may provide for an intuitive interface and robust techniques that help to ensure that geological analyses may be efficient and adaptable to dynamic challenges encountered in the field. Further, as explained, a system may provide for one or more levels of security such that valuable data as to lithology in a field may be protected as to access (e.g., encryption, MFA, etc.) and/or as to understanding (e.g., using codes, etc.).

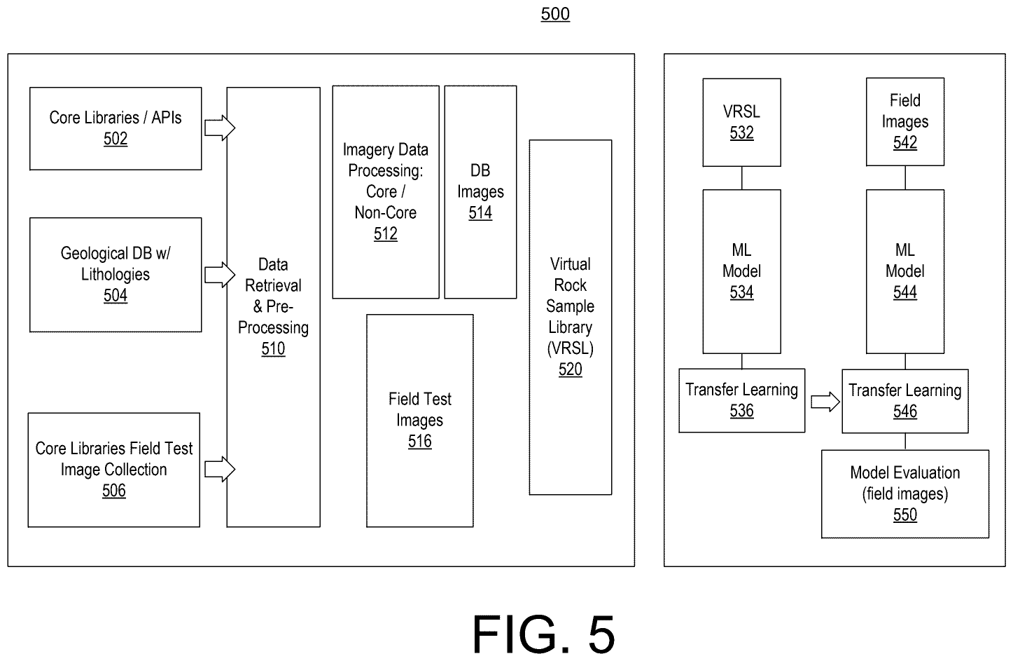

FIG. 5 shows an example of a system 500 that may be utilized for rock characterization. As shown, data may be acquired from one or more sources 502, 504, and 506, which can include, for example, core libraries with accessible via one or more application programming interfaces (APIs), geological databases with lithologies, and core libraries field test image collections, etc. As shown, the system 500 can include a retrieval and pre-processing component such that data may be retrieved and processed. As indicated, the system 500 can provide for generation of pre-processed data for imagery as to cores and non-cores 512, database images 514, and field test images 516, which may provide for generation of a virtual rock sample library (VRSL) 520. As to imagery for cores and non-cores, as an example, one or more image processing techniques may provide for image recognition, segmentation, etc. For example, consider one or more trained machine learning models that may provide for processing of reports. While cores are mentioned, as explained, a system may provide for cuttings assessments, for example, generation of processed data from one or more sources as to cuttings (e.g., cuttings images). While cores and cuttings are mentioned, more generally, a system may provide for rock sample processing where rock sample data may be from one or more sources (e.g., drilling, mining, exploration, etc.).

In the example system 500 of FIG. 5, the VRSL 520 may provide training data 532 (e.g., VRSL data as training data). Such data may provide for generation of a machine learning model (ML model) 534 where characteristics of a trained version of the ML model 534 may be utilized for transfer learning per a transfer learning component 536. As shown, as to implementation of the system 500 for field assessments, field images 542 may provide for generation of an ML model 544, which may be enhanced through transfer learning per a transfer learning component 546, as based on transference of characteristics per the transfer learning component 536. In such an example, the system 500 may provide for model evaluation per a model evaluation component 550, which may provide assessments using field images (e.g., field imagery of rock samples, etc.).

Rock property data may come from the same database or a separate data source such a geological database, core library field test image collection, etc. As an example, lithology may include one or more of sandstone, clay, granite, shale, gneiss, Breccia, siltstone, dolerite, quartzite, schist, basalt, amphibolite, dacite, etc.; noting that there may be hundreds or more of lithologies. As explained, these may be organized as families. As explained, data retrieval and pre-processing components may be implemented. As an example, imagery data processing may provide for indications of core and non-core regions of imagery. As an example, thousands of field test images may be utilized. As an example, locations may be indicated for various samples. For example, consider 2D and/or 3D locations, which may provide for latitude and longitude and, where available, actual depth; noting that depth may be estimated via correlations and/or one or more other techniques.

As shown in the example of FIG. 5, the system 500 may implement a dual transfer learning approach. For example, consider a workflow that includes multiple imagery sources where imagery can be processed using one or more trained ML models (e.g., CNNs, etc.), where processed standardized imagery may be utilized to enhance processing of field images. As explained, dual transfer learning (DTL) may be employed.

As an example, DTL may simultaneously learn marginal and conditional distributions, and exploits the duality between them in a principled way. For example, learning one distribution can help to learn another where such a duality property may lead to mutual reinforcement when adapting both distributions across domains to transfer knowledge. An article by Long et al., entitled Dual Transfer Learning, Proceedings of the 2012 SIAM International Conference on Data Mining (SDM), 2012, 540-551 (10.1137/1.9781611972825.47), is incorporated by reference herein.

As an example, a rock characterization system may use imagery obtained from one environment (domain) and another environment (domain) which is the target use environment (domain). As an example, a rock characterization system may utilize a lab-based domain and a field-based domain where an aim is to improve classification of field-based imagery and, for example, one or more types of associated data (e.g., location, depth, conditions, etc.).

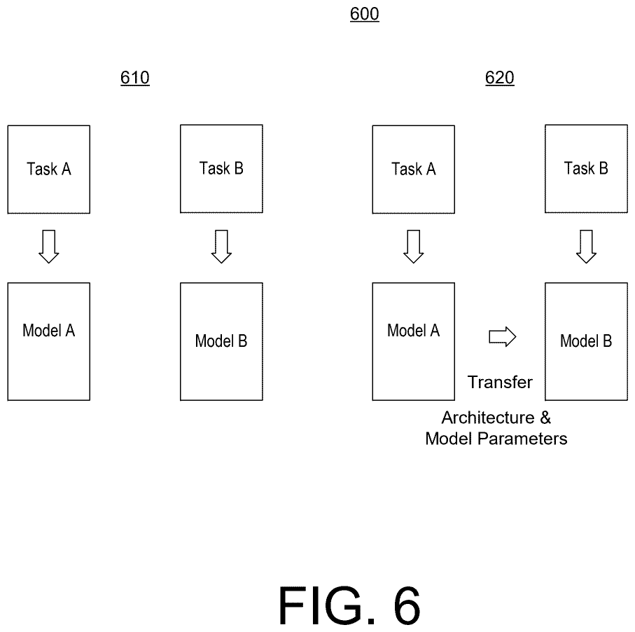

FIG. 6 shows examples of learning techniques 600, one without transfer learning and one with transfer learning. As an example, transfer learning may save time and resources. Various machine learning problems involve training a large amount of data. This type of labeled training data may take more time; whereas, in transfer learning one or more models may be pre-trained, which reduces the size of training data. As an example, transfer learning may improve efficiency of a model while training. Developing machine learning models to solve complex problems can be time-consuming; however, transfer learning may be implemented with a demand to create a model from scratch, for example, it may reuse a developed model by transferring its knowledge.

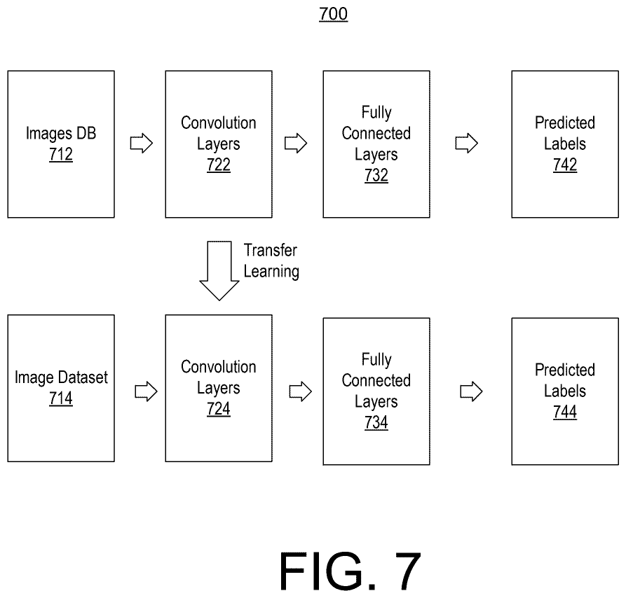

FIG. 7 shows an example of a transfer learning workflow 700. As shown, a first track may involve providing an image database 712, training convolution layers 722 of a first track ML model, training fully connected layers 732 of the first track ML model, and generating predicted labels 742 for images of the image database 712; whereas, a second track may involve providing an image dataset 714, training convolution layers 724 of a second track ML model utilizing characteristics of the convolution layers 722 of the first track via transfer learning, training fully connected layers 734 of the second track ML model, and generating predicted labels 744 for images of the image dataset 714.

An article by Mukhlif et al., entitled Incorporating a Novel Dual Transfer Learning Approach for Medical Images, Sensors 2023, 23, 570. https://doi.org/10.3390/s23020570, is incorporated by reference herein. The article by Mukhlif et al., describes a DTL approach based on the convergence of patterns between source and target domains. The article applies this approach to four pre-trained models using two datasets (skin cancer images and breast cancer images) with fine-tuning the last layers on a sufficient number of unclassified images of the same disease and on a small number of classified images of the target task, in addition to using data augmentation techniques to balance classes and to increase the number of samples. The approach improved the models' performance when fine-tuning was performed on unclassified images of the same disease. As an example, one or more techniques implemented in the article by Mukhlif et al., may be applied in a rock characterization system. For example, the example workflow 700 may implement one or more of the techniques in the article by Mukhlif et al.

As an example, one or more types of ResNet models may be utilized. ResNets are a special type of CNN model with a residual learning structure. Residual (or skip) connections may help to address issues related with training very deep networks. More specifically, skip connections may improve the learning dynamics and error propagation over many layers of non-linear transformations. In such an approach, an identity transform may be used to copy forward and backward signals between sets of non-succeeding layers. Such connections can allow for the training of models with many layers (dozens or hundreds of them). Because each layer in a ResNet model has fewer parameters than was the case with classical models, the total number of parameters remains at a similar level and also the computational complexity.

As explained, a rock characterization system may provide for performing field rock lithology for rock samples (e.g., cores, cuttings, etc.), which may provide for reconstruction of an entire borehole or portion thereof. As explained, a system may provide for comparisons, for example, to one or more other boreholes, etc.

As an example, input to a system can include imagery (e.g., single images, video, etc.) of samples that may be wet and/or dry. As explained, notes, results from one or more tests, a location, video with voice recognition, replay of video with overlay (e.g., augmented reality, annotations, graphical controls, etc.), etc., may be utilized and/or performed. As an example, a system may be robust to allow for real-world applicability in field settings, where conditions such as lighting, texture, and color may vary (e.g., frequency of light, IR, UV, etc.).

As an example, a system may output lithology (e.g., as a prediction, a classification, etc.), confidence, one or more recommendations, a profile, a pieced together image, video, etc. As an example, a system may output structural information such as dip, stratigraphy, etc. As to dip, consider a system that may discern angles of layers in a core sample, which may correspond to dipping layers in a subsurface environment. In various instances, a system may provide for adjustments to imagery where, for example, core portions may be set in a rack at different angles of rotation such that dip angles may appear differently. In such an example, a system may provide for harmonizing angles, for example, given one or more geometric relationships (see, e.g., FIG. 2 as to relationships between a core as a cylinder and a subsurface formation with dipping layers). As an example, dip may be utilized to register one or more layers for one borehole to one or more layers of another borehole that may be a distance away where, for example, depending on distance and direction, dip may be appropriately utilized to adjust depth (e.g., total vertical depth (TVD), etc.). As an example, a system may provide for output of visualizations in 1D, 2D, 3D and/or higher dimensions. As explained, a system may provide for interactions, which may be for corrections, adjustments, etc. As explained, a system may provide for use of one or more ML models, which may be for families and lithologies in families (e.g., in each of the families).

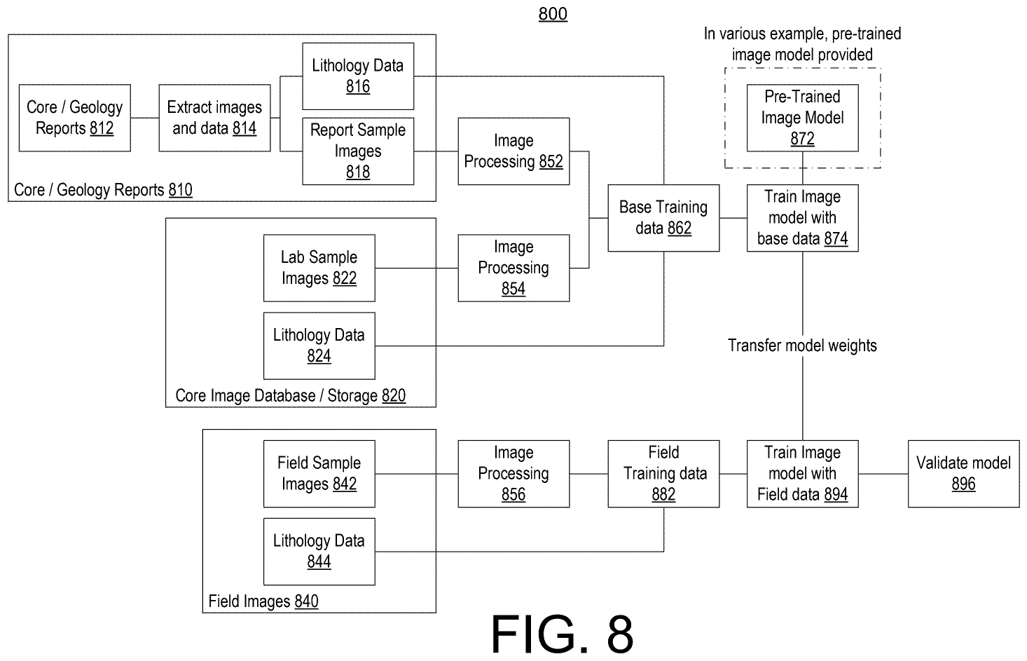

FIG. 8 shows an example of a workflow 800 that includes data sources 810, 820, and 840. As shown, the data source 810 may be a core and geology reports data sources that provides core and geology reports 812 where an extraction block 814 may extract images and data (e.g., using OCR, one or more ML models, etc.), to thereby generate lithology data 816 and report sample images 818. The data source 820 may be a core image database that stores core imagery, which may include lab sample images 822 and lithology data 824. The data source 840 may be a field image data source that provides field sample images 842 and lithology data 844. As shown, the workflow 800 may include multiple tracks, which may include a first track for processing data from one or more of the sources 810 and 820 and a second track for processing data from the source 840, noting that data from the source 840 may provide field images from one or more field sites, etc.

As indicated, the images 818 may be processed via an image processing component 852 and the images 822 may be processed via an image processing component 854 where processed images therefrom may be utilized in combination with the lithology data 816 and 824 to form base training data 862.

In the first track, the base training data 862 may be utilized in a training component 874 to train an image model, which may be based on a pre-trained image model 872 or may be based on a non-pre-trained model. As shown, weights as characteristics of the image model as trained may be transferred. For example, in the second track, an image processing component 856 can provide for processing of the images 842 where results thereof may form, along with the lithology data 844, field training data 882. As shown, the data 822 may be utilized in a training component 894 to train an image model with the field training data 882 in a manner that utilizes transfer learning (e.g., model weights from training of a model of the first track). As shown, the workflow 800 can include a validation component 896 for validating the trained image model as generated by the training component 894. The workflow 800 can provide for generation of a trained ML model that can be utilized for assessing field images of rock samples.

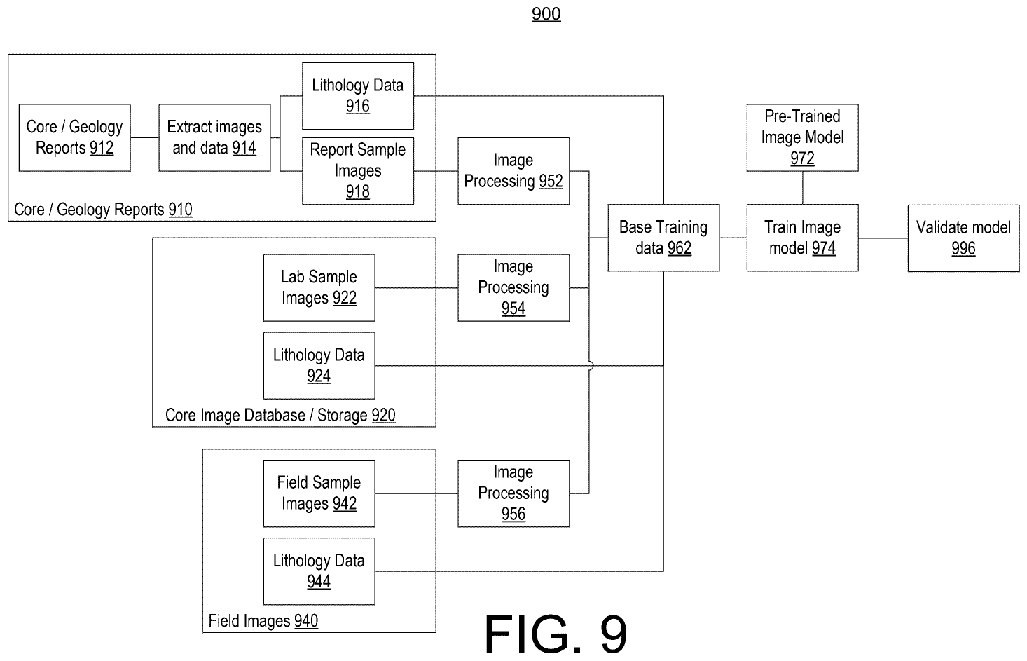

FIG. 9 shows an example of a workflow 900 that includes data sources 910, 920, and 940. As shown, the data source 910 may be a core and geology reports data sources that provides core and geology reports 912 where an extraction block 914 may extract images and data (e.g., using OCR, one or more ML models, etc.), to thereby generate lithology data 916 and report sample images 918. The data source 920 may be a core image database that stores core imagery, which may include lab sample images 922 and lithology data 924. The data source 940 may be a field image data source that provides field sample images 942 and lithology data 944. As shown, the workflow 900 may include a single training track that includes processing data from one or more of the sources 910 and 920 and processing data from the source 940, noting that data from the source 940 may provide field images from one or more field sites, etc.

As indicated, the images 918 may be processed via an image processing component 952 and the images 922 may be processed via an image processing component 954 where processed images therefrom may be utilized in combination with the lithology data 916 and 924 to form part of base training data 962. As shown, an additional part of the base training data 962 can come from an image processing component 956 that can provide for processing of the images 942 where results thereof may form, along with the lithology data 944, the additional part of the base training data 962. As shown, the base training data 962 may be utilized in a training component 974 to train an image model, which may be based on a pre-trained image model 972 or may be based on a non-pre-trained model. As shown, the workflow 900 can include a validation component 996 for validating the trained image model as generated by the training component 974. The workflow 900 can provide for generation of a trained ML model that can be utilized for assessing field images of rock samples.

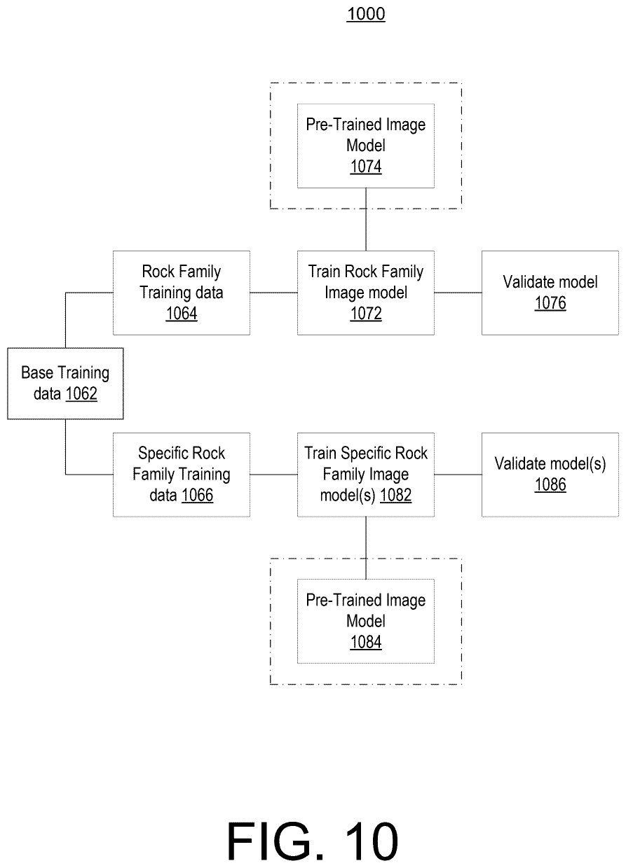

FIG. 10 shows an example of a multi-track workflow 1000 that may be utilized, for example, as to base training data 1062, which may be generated in one or more manners, such as, for example, a manner akin to the base training data 962 of the example workflow 900 of FIG. 9. The workflow 1000 is shown as including tracks for rock family training data 1064 and specific rock family training data 1066 where a training component 1072 can a train rock family image model, which may be based on a pre-trained image model 1074, that can then be validated via a validation component 1076; and where a training component 1082 can train a specific rock family image model, which may be based on a pre-trained image model 1084, that can then be validated via a validation component 1086. While the example of FIG. 10 shows a single specific rock family track, as an example, a number of different, specific rock family tracks may be included. In such an approach, a number of trained models may be generated (e.g., with or without transfer learning) for utilization in assessing rock samples and decision-making (e.g., control, etc.).

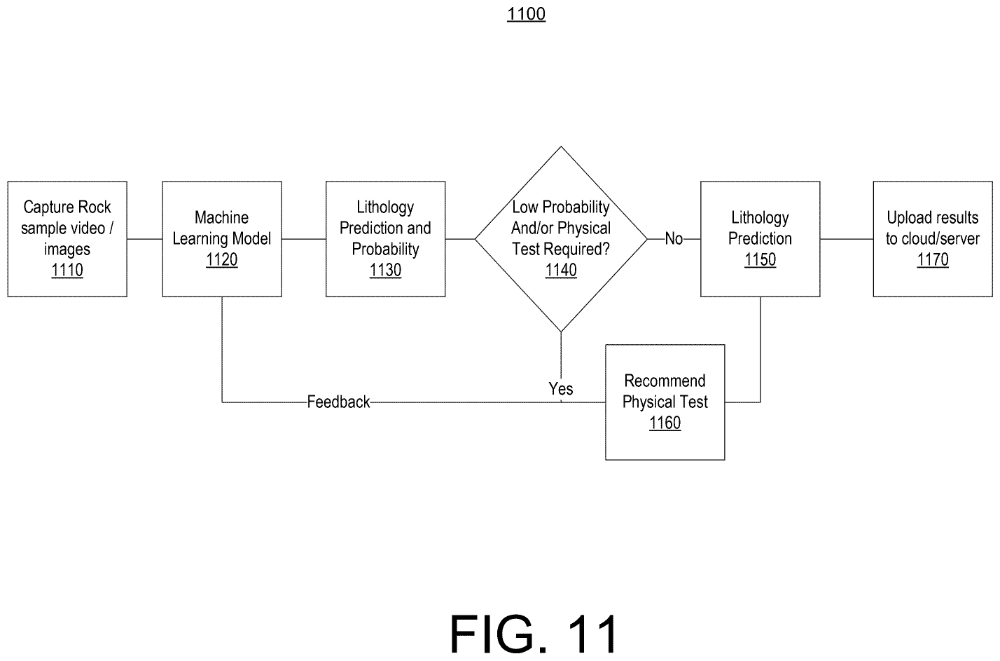

FIG. 11 shows an example of a workflow 1100 that includes a capture block 1110 for capturing rock sample video and/or images, a machine learning block 1120 for training a machine learning model, a prediction block 1130 for lithology prediction and probability, and a decision block 1140 for deciding whether probability is low (e.g., per a threshold) and/or whether a physical test is required (e.g., recommended). As shown, per a “yes” branch of the decision block 1140, a recommendation block 1160 can recommend and/or execute a physical test where results thereof may provide feedback for further training (e.g., re-training) per the machine learning block 1120 and may provide for a lithology prediction 1150. As shown, per a “no” branch of the decision block 1140, the lithology prediction 1150 may be directly generated. As shown, an upload block 1170 may provide for uploading the lithology prediction 1150 as results, which may be to a cloud platform (e.g., a cloud platform server, etc.).

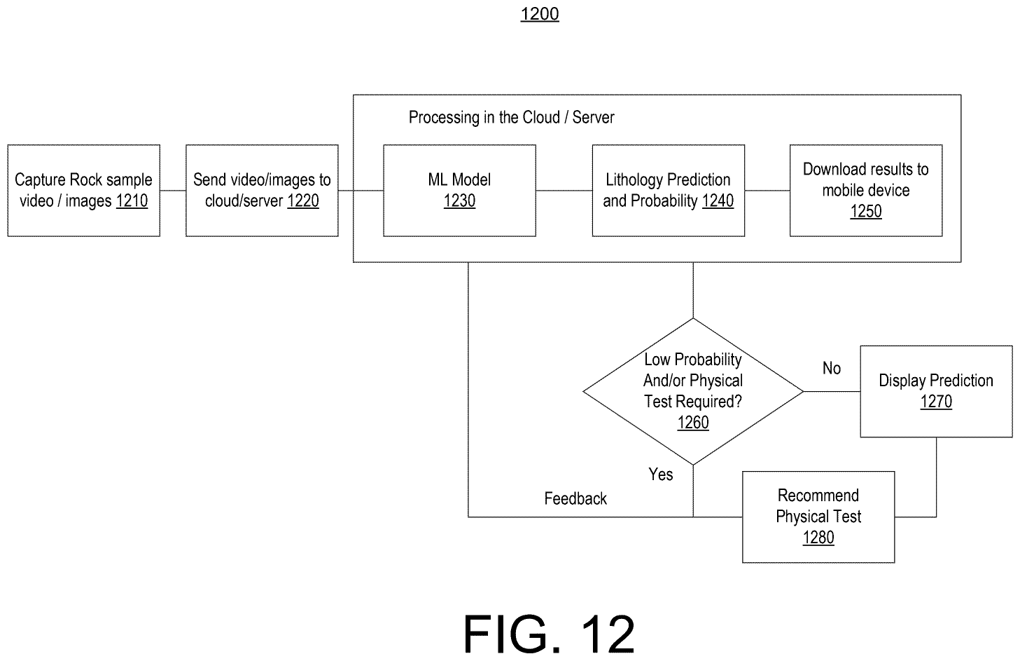

FIG. 12 shows an example of a workflow 1200 that can include a capture block 1210 for capturing rock sample video and/or images. As shown, a send block 1220 may provide for transmitting the video and/or images to a cloud platform (e.g., a cloud platform server, etc.). In the example workflow 1200, processing may be performed using provisioned resources in the cloud platform (e.g., a remote server, etc.). As shown, an ML model 1230 can be implemented for generation of a lithology prediction and probability 1240 where, for example, the results thereof may be downloaded to a mobile device (e.g., at a field site, etc.). While a mobile device is mentioned, as an example, results may be downloaded to field equipment at a site or sites where such results may provide for control of field equipment. As an example, results may be in a machine-readable form, such as, for example, control instructions, commands, etc., that may be executed to cause action or inaction of field equipment.

In the example of FIG. 12, a decision block 1260 may be implemented to assess probability and/or otherwise whether or not a physical test may be required as to one or more rock samples. As shown, per a “yes” branch of the decision block 1260, a recommended physical test 1280 may be performed, called for, executed, etc., where results thereof may provide for feedback in ML model training, re-training, etc., and where results thereof may be rendered to a display 1270, for example, to display a prediction, which may be an ML model-based prediction and/or a physical test prediction (e.g., physical test results). As shown, per a “no” branch of the decision block 1260, the display 1270 may be generated directly.

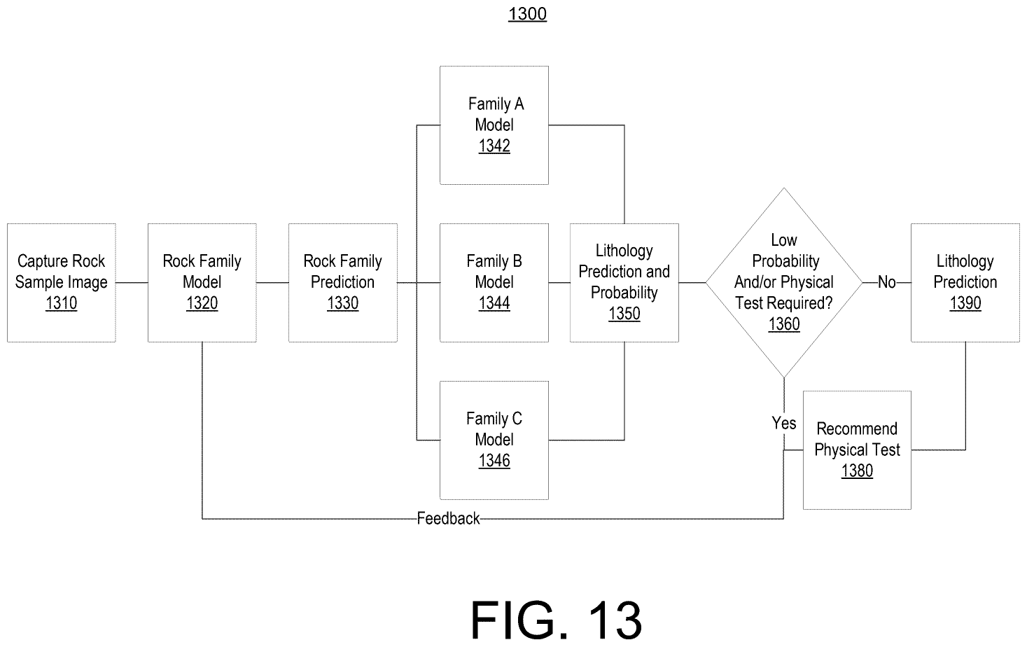

FIG. 13 shows an example of a workflow 1300 that includes a capture block 1310 for capturing rock sample images (e.g., still images, video, etc.), a rock family model block 1320 for generation of a rock family prediction 1330. As shown, the rock family prediction 1330 may provide for directing the workflow 1300 to one or more family model blocks 1342, 1344, and 1346 (e.g., for rock families A, B, and C). As shown, results thereof may provide a lithology prediction and probability 1350. In the example workflow 1300, the rock family model 1320 may be a type of classification model that is trained to generate the rock family prediction 1330 as to a family or families where, as explained, corresponding specific family models 1342, 1344, and 136 may be provided for generation of the lithology prediction and probability 1350.

In the example of FIG. 13, a decision block 1360 may be implemented to assess probability and/or otherwise whether or not a physical test may be required as to one or more rock samples. As shown, per a “yes” branch of the decision block 1360, a recommended physical test 1380 may be performed, called for, executed, etc., where results thereof may provide for feedback in ML model training, re-training, etc., and where results thereof may be output as a lithology prediction 1390, which may be an ML model-based prediction and/or a physical test prediction (e.g., physical test results). As shown, per a “no” branch of the decision block 1360, the lithology prediction 1390 may be generated directly.

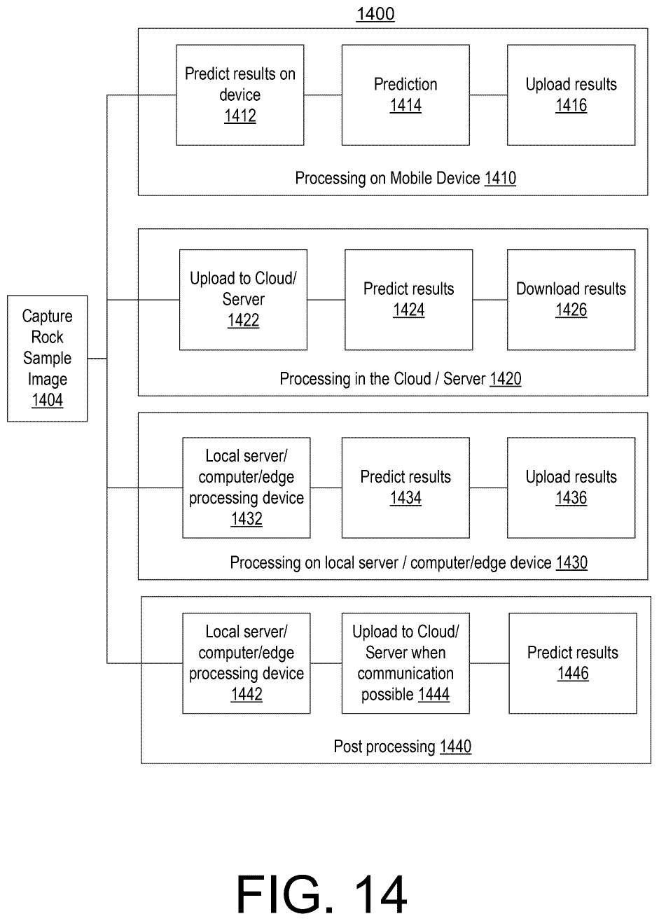

FIG. 14 shows an example system 1400 that includes various options for processing a captured rock sample image 1404 (e.g., imagery, etc.). As shown, processing may be performed on a mobile device 1410; in a cloud platform and/or other remote server 1420; on a local server, computer, edge device 1430, and/or post processing 1440 (e.g., using one or more computational resources).

As shown, for mobile device processing 1410, a prediction block 1412 can provide for generation of a prediction 1414 on the mobile device based on the image 1404 where the prediction may be suitably uploaded for use, storage, etc. As shown, for cloud/server processing 1420, the image 1404 may be uploaded to the cloud/server to predict results 1424 where such results can be downloaded 1426. As shown, for local processing 1430, the image 1404 can be received locally by a processing device 1432 that can predict results 1434 where such results may be uploaded 1436. As shown, for post processing 1440, the image 1404 may be received by a local device 1442 and then uploaded to a remote resource 1444 where or when communication is or becomes possible such that processing may occur to predict results 1446.

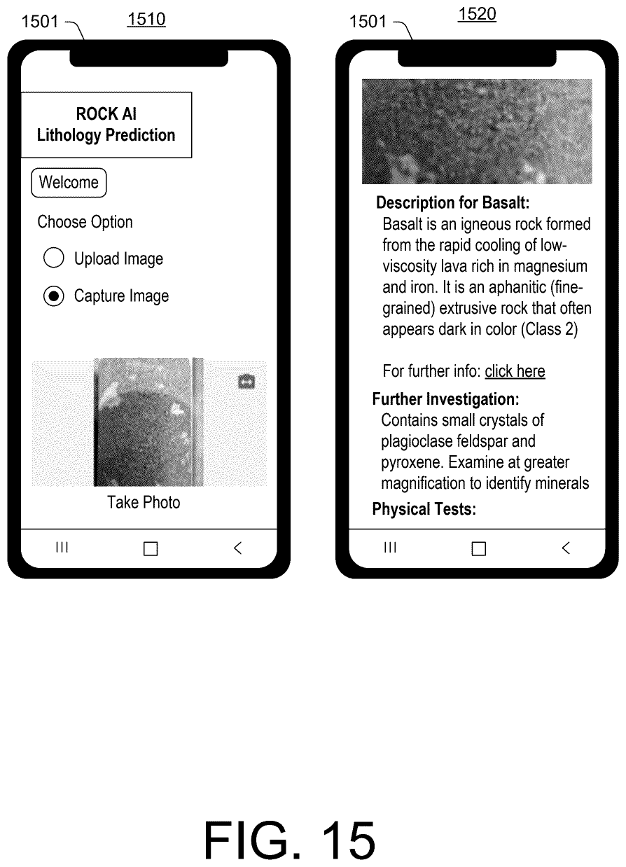

FIG. 15 illustrates examples of graphical user interfaces (GUIs) 1510 and 1520, which may be sequential in that the GUI 1510 transitions to the GUI 1520, for example, responsive to receipt of an image (e.g., rock sample imagery). As shown, the GUIs 1510 and 1520 may be rendered to a display of a mobile device 1501. As shown, the GUI 1510 can provide for options of uploading an image and capturing an image, for example, using a mobile device that includes one or more cameras. As an example, a framework may provide for capturing stereographic imagery, which may be via two or more cameras and/or via imagery captured using two or more perspectives. As an example, a mobile device may provide for generating a 2D and/or a 3D type of image that can provide for assessments of features (e.g., surface roughness, shape, etc.), which may include, for example, features of a rock sample that may be prior to testing, during testing, and/or after testing (e.g., of one or more tests). In the example of FIG. 15, the GUI 1510 can display a captured image of a rock sample to a display, which may be a raw and/or a processed image. As an example, one or more machine learning models may be employed for image enhancement, segmentation, classification, etc. As explained, a framework may provide for prediction of lithology of a rock sample. For example, the GUI 1520 indicates that the rock sample of the GUI 1510 is likely basalt where further information may be acquired (e.g., via a customized link, etc.) and/or one or more physical tests performed on the rock sample, for example, to verify, increase likelihood, etc.

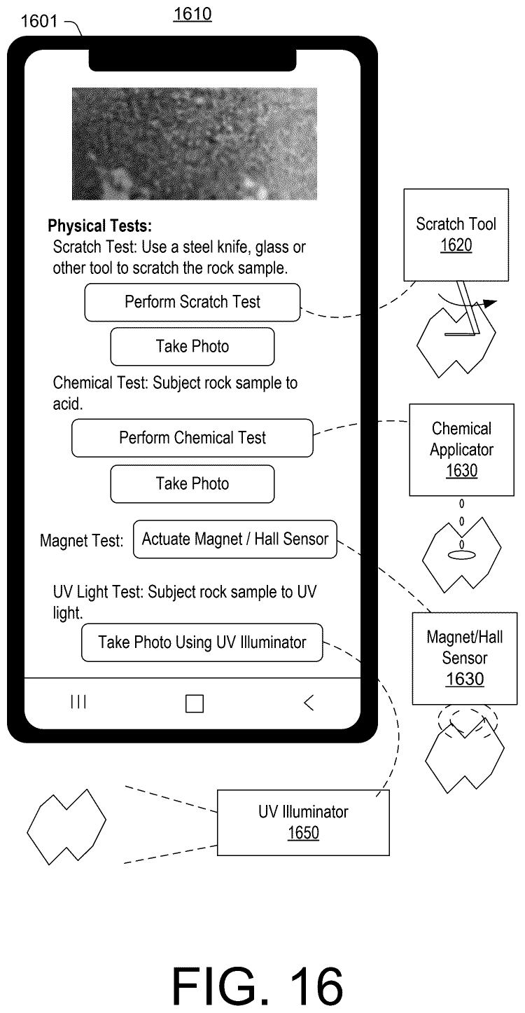

FIG. 16 shows an example of a GUI 1610 that may pertain to one or more physical tests that may be rendered to a display of a mobile device 1601. For example, consider a scratch test that may involve scratching a rock sample and/or using the rock sample to scratch another material. In such an example, one or more characteristics may be determined, which may include, for example, hardness (e.g., consider Shore hardness according to a scale). As an example, a scratch tool 1620 may be in communication with the mobile device for performing a scratch test where the scratch tool 1620 may be actuated using a graphical control of the GUI 1610. As an example, a chemical applicator 1630 may be in communication with the mobile device for performing a chemical test (e.g., an acid test, etc.) where the chemical applicator 1630 may be actuated using a graphical control of the GUI 1610. As an example, a magnet and/or a Hall sensor 1630 may be in communication with the mobile device for performing a magnetic test where the magnet and/or the Hall sensor 1630 may be actuated using a graphical control of the GUI 1610. As an example, a UV illuminator 1640 may be in communication with the mobile device for performing a UV light test where the UV illuminator 1640 may be actuated using a graphical control of the GUI 1610.

As to the magnet and/or the Hall sensor 1630, consider utilization of a Hall sensor that may be located a distance from a rock sample and a magnet (e.g., permanent, electromagnet, etc.) that may provide one or more magnetic fields with respect to one or more regions of the rock sample. In such an example, another Hall sensor may be arranged in a gradiometric configuration to cancel background signal applied by the magnet and reduce noise. As an example, circuitry may provide for biasing one or more sensors and allowing adjustments to background signal cancelation, as appropriate (see, e.g., Araujo et al., 2019). In such an example, one or more characteristics of a rock sample may be assessed. As an example, a characteristic may relate to past magnetic fields by analyzing magnetizations of a rock sample. As an example, a magnet test may provide for determining whether or not a rock sample includes one or more types of magnetic materials. As an example, a magnet test may employ magnetic microscope technology, as a type of magnetometer, to generate a magnetic field map of a rock sample (e.g., with a spatial resolution of the order of millimeters or less). As an example, a magnet test may provide for distinguishing different magnetic minerals, whether from a single rock sample or from multiple rock samples.

As an example, a framework may provide for test recommendation, test performance, test result assessment, etc. As an example, test equipment may be integrated into a mobile device or other device. As an example, communication between a computing device and test equipment may be via wire, fiber, wireless technology, etc. As an example, a mobile device may include a camera or cameras that may provide for observing testing, acquiring test results, etc. As an example, an acquired image or video may be assessed to determine whether further testing or re-testing may be beneficial. For example, consider a chemical test where an image may be assessed to determine whether the chemical test was successful or not. In such an example, if a pH of an acid may be insufficient for a test, an image may reveal such information and request that a lower pH acid be applied to a rock sample. While UV illumination is mentioned in the example of FIG. 16, as an example, another type of illumination may be recommended, which may include IR, visible, etc., where a mobile device or other machine vision equipment may provide for such illumination and/or image capture at a suitable wavelength (e.g., per direct illumination, fluorescence, etc.). As explained, a framework may provide for dynamic assessment of a rock sample where one or more tests may be recommended that may provide for increased accuracy, knowledge, etc., which, in turn, may be provided as feedback for one or more purposes (e.g., ML model training, equipment control, etc.).

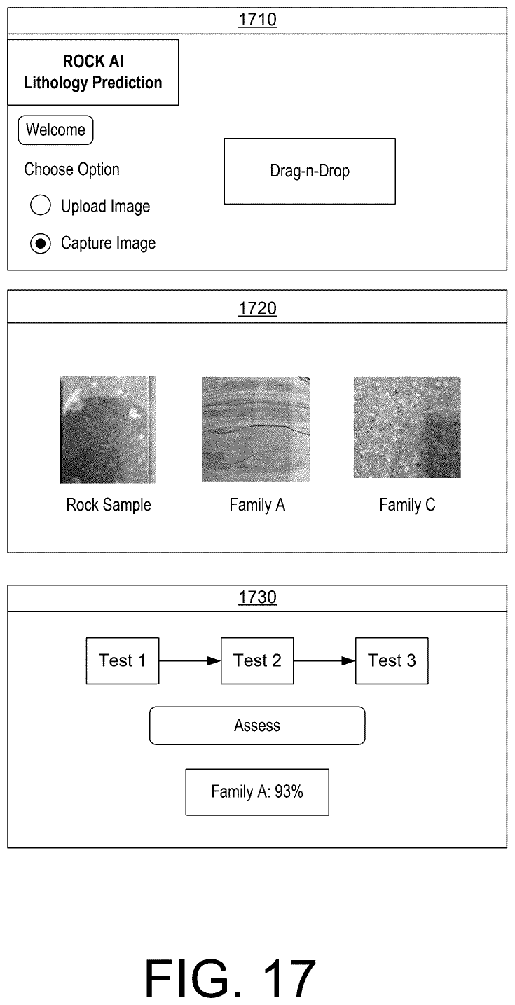

FIG. 17 shows examples of GUls 1710, 1720, and 1730, which may be rendered to one or more display and which may be sequential and transition from one to another responsive to interaction via human and/or machine. As shown, the GUI 1710 can include options for lithology prediction, which may include upload, capture, and/or file drag-n-drop options. As shown, the GUI 1720 may be rendered responsive to receipt of a rock sample image, for example, to provide for a number of predictions, which may be organized by rock family. For example, consider possible families as Family A and Family C. In such an example, a framework may provide for test recommendation, which may recommend one or more tests to improve prediction. For example, consider a number of tests organized in a sequence that aim to increase accuracy of a prediction. In such an example, after a first test, an assessment may be made to determine whether a prediction is sufficiently accurate. If not, then a second test may be performed, and similarly, as appropriate, one or more additional tests. In such an example, after each test, a framework may dynamically (e.g., automatically) provide an updated prediction and/or prediction accuracy (e.g., probability). As an example, a user may perform a test or tests until a desired level of prediction accuracy is achieved. As an example, a result or results may be utilized as feedback, for example, to train an ML model, retrain an ML model, control equipment, etc.

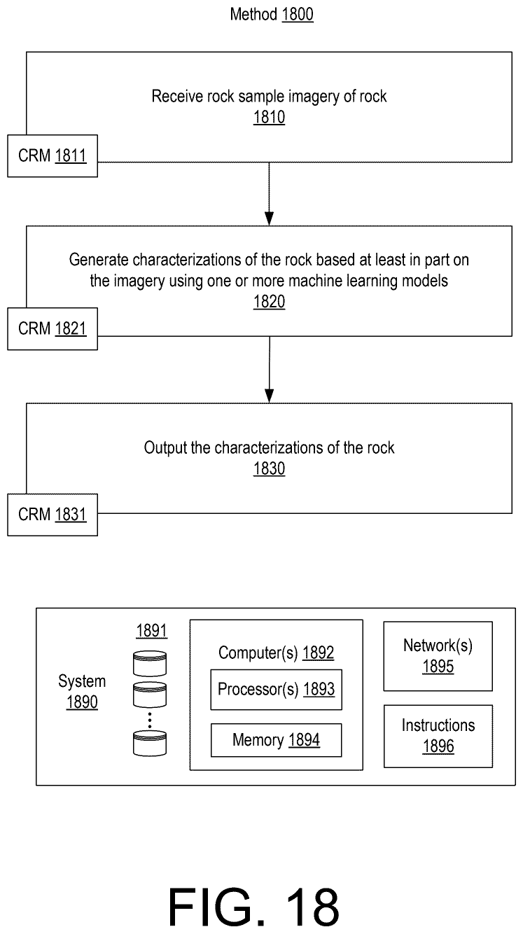

FIG. 18 shows an example of a method 1800 that includes a reception block 1810 for receiving rock sample imagery; a generation block 1820 for generating characterizations of the rock based at least in part on the rock sample imagery using one or more machine learning models; and an output block 1830 for outputting the characterizations of the rock.

FIG. 18 also shows various computer-readable media (CRM) blocks 1811, 1821, and 1831. Such blocks may include instructions that are executable by one or more processors, which may be one or more processors of a computational framework, a system, a computer, etc. A computer-readable medium may be a computer-readable storage medium that is not a signal, not a carrier wave and that is non-transitory. For example, a computer-readable medium may be a physical memory component that may store information in a digital format.

In the example of FIG. 18, a system 1890 includes one or more information storage devices 1891, one or more computers 1892, one or more networks 1895 and instructions 1896. As to the one or more computers 1892, each computer may include one or more processors (e.g., or processing cores) 1893 and a memory 1894 for storing the instructions 1896, for example, executable by at least one of the one or more processors. As an example, a computer may include one or more network interfaces (e.g., wired or wireless), one or more graphics cards, a display interface (e.g., wired or wireless), etc. The system 1890 may be specially configured to perform one or more portions of the method 1800 of FIG. 18.

As an example, a system may employ one or more machine learning models. For example, consider one or more trained machine learning models that may provide for receiving imagery and outputting one or more characteristics of rock, which may be physical characteristics. For example, consider one or more of rock type (e.g., with classification), color, texture (e.g., grain size, roundness, sorting), cement and/or matrix material, fossils, sedimentary structures, porosity, etc.

As an example, output of a system may be in the form of one or more logs, which may include an estimate of percentage of each rock type, an interpretation of lithology, presence and/or type/composition of hydrocarbons, minerals, etc.

As an example, a system may provide for generation of training data, which may be utilized for one or more of training, testing, tuning, etc., one or more machine learning models. As an example, a system may provide for generation of imagery that can be suitably high in contrast. Such imagery may provide for robust training to allow for generation of a trained machine learning model that can improve automation, consistency, etc.

As an example, a system may provide for processing of individual images, video and/or streaming video. As an example, a system may provide for post-processing and/or real-time processing. For example, consider capturing imagery and then processing responsive to demand, capture of an amount of data, passage of an amount of time, coverage of imagery (e.g., spatially, temporally, etc.), etc. As to real-time processing, processing may occur as imagery is captured where results thereof may be generated in real-time (e.g., with computational and/or transmission latency), where results may be utilized for one or more purposes (e.g., decision-making, control, etc.).

As an example, one or more system architectures may be implemented. As explained, an architecture may provide for fine-tuning a pre-trained image model, for example, where or when sufficient training data for a target application or environment are available. As explained, an architecture may provide for training from scratch (e.g., not using a pre-trained model) an image model, for example, where or when a sufficient amount of training data for a target application or environment are available. As an example, an architecture may provide for fine-tuning a pre-trained image model with images from a first environment and using transfer learning for fine-tuning with images from a second environment (e.g., where a larger number of images exist from the first environment).

As an example, a system may provide for assessments as to one or more types of rock samples, which may include one or more of cores, cuttings, millings, etc. As explained, in various instances human-made material may be assessed, for example, where a machine component breaks such that a core, a cutting, etc., may include a chunk of human-made material (e.g., metal, plastic, ceramic, etc.). As explained, imagery may include imagery of exposed surface formations and/or rocks.

As explained, one or more types of models may be utilized. For example, consider an ImageNet model, a ResNet model, a VGG model, etc. As an example, a model may be or include a CNN that may be trainable or pre-trained. For example, consider the VGG-16 model trained using suitable resources (e.g., libraries, databases, hyperparameter tuning techniques, etc.).

The VGG-16 model is a convolutional neural network (CNN) architecture of the Visual Geometry Group (VGG) at the University of Oxford. The VGG-16 model is characterized by its depth, consisting of 16 layers, including 13 convolutional layers and 3 fully connected layers. VGG-16 has demonstrated performance on computer vision tasks, including image classification and object recognition. The VGG-16 model architecture features a stack of convolutional layers followed by max-pooling layers, with progressively increasing depth. This design enables the VGG-16 model to learn intricate hierarchical representations of visual features in deep learning applications.

As an example, one or more image analysis machine learning models may be employed. As an example, a U-Net type of machine learning model may be employed. U-Net is an architecture that may be employed for various tasks (e.g., semantic segmentation, etc.). It can include a contracting path and an expansive path where the contracting path follows an architecture of a convolutional network. It may provide for repeated application of two 3×3 convolutions (unpadded convolutions), each followed by a rectified linear unit (ReLU) and a 2×2 max pooling operation with stride 2 for downsampling. In such an example, at each downsampling step it may be possible to double the number of feature channels. As an example, every step in an expansive path may provide for an upsampling of a feature map followed by a 2×2 convolution (“up-convolution”) that halves the number of feature channels, a concatenation with the correspondingly cropped feature map from the contracting path, and two 3×3 convolutions, each followed by a ReLU. As an example, cropping may be employed due to loss of border pixels in each convolution. As an example, at a final layer, a 1×1 convolution may be used to map each 64-component feature vector to the desired number of classes. As an example, a total network may include 23 convolutional layers.

As an example, a system may include one or more models for imagery, text, etc. As an example, a system may include one or more models for handling of time series data. As explained, a system may provide for control of one or more field operations. For example, consider control of drilling operations using assessed imagery of cuttings as may be extracted from mud. In such an example, one or more models may provide for assessing the imagery of cuttings while one or more other models may provide for assessing drilling operations that generate the cuttings. As explained, cuttings may relate to bit wear, mud properties, mud flowrate, etc., which may vary over time in a manner responsive to equipment operations and/or equipment and formation (e.g., rock, fluid, etc.) interactions. For example, consider a system where drillstring friction during rotation and/or translation of a drillstring in a borehole may be represented using time series data and where cuttings may be assessed using imagery data where, for example, control actions may be generated using multiple models to control drilling parameters as to friction, which may relate to cutting shape, size, etc., and/or hole cleaning by flow of mud (e.g., carrying cuttings to surface).

As explained, assessment of rock samples may be part of a workflow for control of a robot (e.g., ground, sea, air, etc.). In such an example, one or more types of models may provide for control of robotic motion, which, in turn, may depend on assessment of rock sample imagery, which may be assessed using one or more types of models that may include one or more types of models that differ from those for control of robotic motion. In such an example, a system may implement heterogenous models.

As to types of machine learning models, consider one or more of a support vector machine (SVM) model, a k-nearest neighbors (KNN) model, an ensemble classifier model, a neural network (NN) model, etc. As an example, a machine learning model may be a deep learning model (e.g., deep Boltzmann machine, deep belief network, convolutional neural network, stacked auto-encoder, etc.), an ensemble model (e.g., random forest, gradient boosting machine, bootstrapped aggregation, AdaBoost, stacked generalization, gradient boosted regression tree, etc.), a neural network model (e.g., radial basis function network, perceptron, back-propagation, Hopfield network, etc.), a regularization model (e.g., ridge regression, least absolute shrinkage and selection operator, elastic net, least angle regression), a rule system model (e.g., cubist, one rule, zero rule, repeated incremental pruning to produce error reduction), a regression model (e.g., linear regression, ordinary least squares regression, stepwise regression, multivariate adaptive regression splines, locally estimated scatterplot smoothing, logistic regression, etc.), a Bayesian model (e.g., naïve Bayes, average on-dependence estimators, Bayesian belief network, Gaussian naïve Bayes, multinomial naïve Bayes, Bayesian network), a decision tree model (e.g., classification and regression tree, iterative dichotomiser 3, C4.5, C5.0, chi-squared automatic interaction detection, decision stump, conditional decision tree, M5), a dimensionality reduction model (e.g., principal component analysis, partial least squares regression, Sammon mapping, multidimensional scaling, projection pursuit, principal component regression, partial least squares discriminant analysis, mixture discriminant analysis, quadratic discriminant analysis, regularized discriminant analysis, flexible discriminant analysis, linear discriminant analysis, etc.), an instance model (e.g., k-nearest neighbor, learning vector quantization, self-organizing map, locally weighted learning, etc.), a clustering model (e.g., k-means, k-medians, expectation maximization, hierarchical clustering, etc.), etc.

As an example, a machine model, which may be a machine learning model (ML model), may be built using a computational framework with a library, a toolbox, etc., such as, for example, those of the MATLAB framework (MathWorks, Inc., Natick, Massachusetts). The MATLAB framework includes a toolbox that provides supervised and unsupervised machine learning algorithms, including support vector machines (SVMs), boosted and bagged decision trees, k-nearest neighbor (KNN), k-means, k-medoids, hierarchical clustering, Gaussian mixture models, and hidden Markov models. Another MATLAB framework toolbox is the Deep Learning Toolbox (DLT), which provides a framework for designing and implementing deep neural networks with algorithms, pretrained models, and apps. The DLT provides convolutional neural networks (ConvNets, CNNs) and long short-term memory (LSTM) networks to perform classification and regression on image, time-series, and text data. The DLT includes features to build network architectures such as generative adversarial networks (GANs) and Siamese networks using custom training loops, shared weights, and automatic differentiation. The DLT provides for model exchange various other frameworks.

As an example, the TENSORFLOW framework (Google LLC, Mountain View, CA) may be implemented, which is an open-source software library for dataflow programming that includes a symbolic math library, which may be implemented for machine learning applications that may include neural networks. As an example, the CAFFE framework may be implemented, which is a DL framework developed by Berkeley AI Research (BAIR) (University of California, Berkeley, California). As another example, consider the SCIKIT platform (e.g., scikit-learn), which utilizes the PYTHON programming language. As an example, a framework such as the APOLLO AI framework may be utilized (APOLLO.AI GmbH, Germany). As an example, a framework such as the PYTORCH framework may be utilized (Facebook AI Research Lab (FAIR), Facebook, Inc., Menlo Park, California).

As an example, a training method may include various actions that may operate on a dataset to train a ML model. As an example, a dataset may be split into training data and test data where test data may provide for evaluation. A method may include cross-validation of parameters and best parameters, which may be provided for model training.

The TENSORFLOW framework may run on multiple CPUs and GPUs (with optional CUDA (NVIDIA Corp., Santa Clara, California) and SYCL (The Khronos Group Inc., Beaverton, Oregon) extensions for general-purpose computing on graphics processing units (GPUs)). TENSORFLOW is available on 64-bit LINUX, MACOS (Apple Inc., Cupertino, California), WINDOWS (Microsoft Corp., Redmond, Washington), and mobile computing platforms including ANDROID (Google LLC, Mountain View, California) and IOS (Apple Inc.) operating system-based platforms.

TENSORFLOW computations may be expressed as stateful dataflow graphs; noting that the name TENSORFLOW derives from the operations that such neural networks perform on multidimensional data arrays. Such arrays may be referred to as “tensors”.

As an example, a device may utilize TENSORFLOW LITE (TFL) or another type of lightweight framework. TFL is a set of tools that enables on-device machine learning where models may run on mobile, embedded, and IoT devices. TFL is optimized for on-device machine learning, by addressing latency (no round-trip to a server), privacy (no personal data leaves the device), connectivity (Internet connectivity is demanded), size (reduced model and binary size) and power consumption (e.g., efficient inference and a lack of network connections). TFL includes multiple platform support, covering ANDROID and iOS devices, embedded LINUX, and microcontrollers and diverse language support, which includes JAVA, SWIFT, Objective-C, C++, and PYTHON. TFL provides for high performance, with hardware acceleration and model optimization.

As an example, a method can include receiving rock sample imagery; generating characterizations of the rock based at least in part on the imagery using one or more machine learning models; and outputting the characterizations of the rock. In such an example, the receiving, the generating and the outputting may be performed by a mobile device at a field site where a rock sample is cut (at surface and/or downhole) and imaged to generate the rock sample imagery. In such an example, the mobile device may not be in communication with a communication network that extends to a site remote from the field site. For example, the mobile device may include features that allow for local generation and output at a field site. In various examples, a mobile device may provide for transmission of rock sample imagery to a remote site upon establishing a communication network connection (e.g., via cellular, satellite, etc.).

As an example, generating characterizations of rock may generate confidence metrics for the characterizations of the rock (e.g., probabilities, etc.). In such an example, responsive to one of the confidence metrics being less than a confidence metric criterion, a method may include generating a recommendation that aims to increase confidence. In such an example, the recommendation may be for or include a test recommendation for performing a test on at least a portion of a rock sample at a field site.

As an example, generating characterizations of certain rock types may be difficult due to similar physical appearance. In such an example, a method may include generating a recommendation for performing tests on a portion of the rock sample in the field to evaluate the physical characteristics (e.g., shape, size, chemical, etc.) can help to differentiate between the different rock types.

As an example, a method may include outputting characterizations of rock by rendering a graphical user interface to a display device. In such an example, the graphical user interface may include at least a portion of core sample imagery and one or more graphics indicative of one or more of the characterizations of the rock. As an example, core sample imagery may be real-time live imagery where, for example, a user points a mobile device at a core and where imagery of the core is processed to generate characterizations of rock in real-time or near real-time. As explained, a machine learning model may be a relatively lightweight ML model, as may be implemented using a framework such as the TFL framework, etc.

As an example, rock sample imagery may be acquired using equipment at a field site where a method may include receiving and generating that are performed using equipment remote from the field site. In such an example, the equipment at the field site may be or include a mobile phone and, for example, the equipment remote from the field site may be or include cloud-based equipment. In such an example, outputting may be performed using the equipment at the field site responsive to establishment of a communication network connection between the equipment remote from the field site and the equipment at the field site.

As an example, a method may include generating characterizations of rock by, at least in part, implementing transfer learning. In such an example, transfer learning may include a first domain that utilizes a first set of rock sample imagery and a second domain that utilizes a different set of rock sample imagery (e.g., core sample imagery, cuttings sample imagery, etc.). In such an example, transfer learning may transfer knowledge from the first set of imagery, as processed via a first one of one or more machine learning models, to a second one of the one or more machine learning models. As an example, a model may be first trained with a larger dataset. In such an example, transfer learning may be implemented as a process where model weights are taken from the model and then used as a starting point for a second model which is then trained with a smaller data set. For example, consider use of a large number of lab images and smaller number of field images.

As an example, transfer learning can transfer knowledge from a first one of one or more machine learning models, trained on a first set of imagery, to a second one of one or more machine learning models that is further trained on the second set of images.

As an example, rock sample imagery may be or include imagery from a field site associated with mineral exploration. In such an example, the field site may not have associated seismic survey data. As an example, exploration for oil and/or gas may involve seismic imaging via a seismic survey to generate seismic survey data. In such an example, the seismic survey data may provide for determinations as to layers of a subsurface region, which may facilitate assessments of rock samples. For example, where interpretations of seismic survey data are available, they may be utilized to generate characterizations of rock (e.g., consider an approach where a ML model receives information based on seismic survey data). As explained, a rock characterization system may operate using input that may not include input based on seismic survey data, for example, consider a mining exploration workflow for a region where a seismic survey has not been performed for that region. Without seismic survey data, characterization of rock can become more challenging.

As an example, a method may include receiving data and generating characterizations of rock based at least in part on the data and based at least in part on core sample imagery. In such an example, data may include one or more types of non-core sample imagery, for example, consider location data, environmental conditions data, test equipment data, voice recognition data generated by utterances received by a microphone of a mobile device, data received via a GUI, etc.

As an example, a system can include a processor; memory operatively coupled to the processor; a network interface; and processor-executable instructions stored in the memory to instruct the system to: receive rock sample imagery; generate characterizations of the rock based at least in part on the imagery using one or more machine learning models; and output the characterizations of the rock.

As an example, one or more computer-readable storage media can include computer-executable instructions executable to instruct a computing system to: receive rock sample imagery; generate characterizations of the rock based at least in part on the imagery using one or more machine learning models; and output the characterizations of the rock.

As explained, a system may include one or more machine learning models, which may be resident locally and/or remotely. As example, a system may include a lightweight machine learning framework (e.g., TFL, etc.). As an example, a method may employ one or more machine learning models.

As an example, a method may be implemented in part using computer-readable media (CRM), for example, as a module, a block, etc. that include information such as instructions suitable for execution by one or more processors (or processor cores) to instruct a computing device or system to perform one or more actions. As an example, a single medium may be configured with instructions to allow for, at least in part, performance of various actions of a method. As an example, a computer-readable medium (CRM) may be a computer-readable storage medium (e.g., a non-transitory medium) that is not a carrier wave. As an example, a computer-program product may include instructions suitable for execution by one or more processors (or processor cores) where the instructions may be executed to implement at least a portion of a method or methods.

According to an embodiment, one or more computer-readable media may include computer-executable instructions to instruct a computing system to output information for controlling a process. For example, such instructions may provide for output to sensing process, an injection process, drilling process, an extraction process, an extrusion process, a mining process, a pumping process, a heating process, etc.

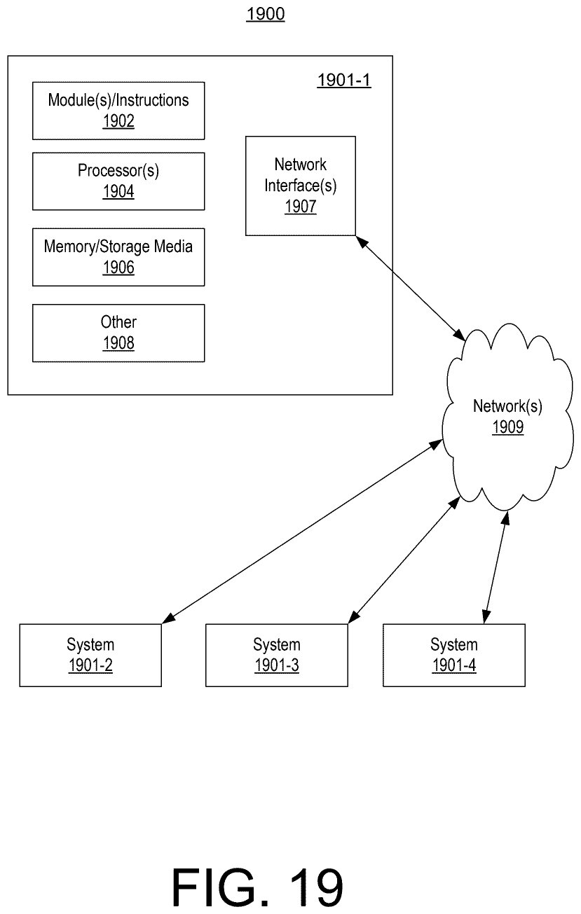

In some embodiments, a method or methods may be executed by a computing system. FIG. 19 shows an example of a system 1900 that may include one or more computing systems 1901-1, 1901-2, 1901-3 and 1901-4, which may be operatively coupled via one or more networks 1909, which may include wired and/or wireless networks.

As an example, a system may include an individual computer system or an arrangement of distributed computer systems. In the example of FIG. 19, the computer system 1901-1 may include one or more modules 1902, which may be or include processor-executable instructions, for example, executable to perform various tasks (e.g., receiving information, requesting information, processing information, simulation, outputting information, etc.).

As an example, a module may be executed independently, or in coordination with, one or more processors 1904, which is (or are) operatively coupled to one or more storage media 1906 (e.g., via wire, wirelessly, etc.). As an example, one or more of the one or more processors 1904 may be operatively coupled to at least one of one or more network interface 1907. In such an example, the computer system 1901-1 may transmit and/or receive information, for example, via the one or more networks 1909 (e.g., consider one or more of the Internet, a private network, a cellular network, a satellite network, etc.). As shown, one or more other components 1908 may be included in the computer system 1901-1.

As an example, the computer system 1901-1 may receive from and/or transmit information to one or more other devices, which may be or include, for example, one or more of the computer systems 1901-2, etc. A device may be located in a physical location that differs from that of the computer system 1901-1. As an example, a location may be, for example, a processing facility location, a data center location (e.g., server farm, etc.), a rig location, a wellsite location, a downhole location, etc.

As an example, a processor may be or include a microprocessor, microcontroller, processor module or subsystem, programmable integrated circuit, programmable gate array, or another control or computing device.

As an example, the storage media 1906 may be implemented as one or more computer-readable or machine-readable storage media. As an example, storage may be distributed within and/or across multiple internal and/or external enclosures of a computing system and/or additional computing systems.

As an example, a storage medium or storage media may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories, magnetic disks such as fixed, floppy and removable disks, other magnetic media including tape, optical media such as compact disks (CDs) or digital video disks (DVDs), BLUERAY disks, or other types of optical storage, or other types of storage devices.

As an example, a storage medium or media may be located in a machine running machine-readable instructions, or located at a remote site from which machine-readable instructions may be downloaded over a network for execution.

As an example, various components of a system such as, for example, a computer system, may be implemented in hardware, software, or a combination of both hardware and software (e.g., including firmware), including one or more signal processing and/or application specific integrated circuits.

As an example, a system may include a processing apparatus that may be or include a general-purpose processors or application specific chips (e.g., or chipsets), such as ASICs, FPGAs, PLDs, or other appropriate devices.

As an example, a device may be a mobile device that includes one or more network interfaces for communication of information. For example, a mobile device may include a wireless network interface (e.g., operable via IEEE 802.11, ETSI GSM, BLUETOOTH, satellite, etc.). As an example, a mobile device may include components such as a main processor, memory, a display, display graphics circuitry (e.g., optionally including touch and gesture circuitry), a SIM slot, audio/video circuitry, motion processing circuitry (e.g., accelerometer, gyroscope), wireless LAN circuitry, smart card circuitry, transmitter circuitry, GPS circuitry, and a battery. As an example, a mobile device may be configured as a cell phone, a tablet, etc. As an example, a method may be implemented (e.g., wholly or in part) using a mobile device. As an example, a system may include one or more mobile devices.

As an example, a system may be a distributed environment, for example, a so-called “cloud” environment where various devices, components, etc. interact for purposes of data storage, communications, computing, etc. As an example, a device or a system may include one or more components for communication of information via one or more of the Internet (e.g., where communication occurs via one or more Internet protocols), a cellular network, a satellite network, etc. As an example, a method may be implemented in a distributed environment (e.g., wholly or in part as a cloud-based service).

As an example, information may be input from a display (e.g., consider a touchscreen), output to a display or both. As an example, information may be output to a projector, a laser device, a printer, etc. such that the information may be viewed. As an example, information may be output stereographically or holographically. As to a printer, consider a 2D or a 3D printer. As an example, a 3D printer may include one or more substances that may be output to construct a 3D object. For example, data may be provided to a 3D printer to construct a 3D representation of a subterranean formation. As an example, layers may be constructed in 3D (e.g., horizons, etc.), geobodies constructed in 3D, etc. As an example, holes, fractures, etc., may be constructed in 3D (e.g., as positive structures, as negative structures, etc.).

Although only a few examples have been described in detail above, those skilled in the art will readily appreciate that many modifications are possible in the examples. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

### BIBLIOGRAPHY

### Documents Incorporated by Reference Herein

Mukhlif, Abdulrahman Abbas, Belal Al-Khateeb, and Mazin Abed Mohammed. 2023. “Incorporating a Novel Dual Transfer Learning Approach for Medical Images” Sensors 23, no. 2:570. https://doi.org/10.3390/s23020570.
An, G., Akiba, M., Omodaka, K. et al. Hierarchical deep learning models using transfer learning for disease detection and classification based on small number of medical images. Sci Rep 11, 4250 (2021). https://doi.org/10.1038/s41598-021-83503-7.
Alzubaidi, Fatimah & Mostaghimi, Peyman & Swietojanski, Pawel & Clark, Stuart & Armstrong, Ryan. (2020). Automated lithology classification from drill core images using convolutional neural networks. Journal of Petroleum Science and Engineering. 197. 107933. 10.1016/j.petrol.2020.107933.
Tolstaya, E., Shakirov, A., and M. Mezghani. “Lithology Prediction from Drill Cutting Images Using Convolutional Neural Networks and Automated Dataset Cleaning.” Paper presented at the ADIPEC, Abu Dhabi, UAE, October 2023. doi: https://doi.org/10.2118/216418-MS.
Araujo, J. F. D. F., Reis, A. L. A., Oliveira, V. C., Jr., Santos, A. F., Luz-Lima, C., Yokoyama, E., Mendoza, L. A. F., Pereira, J. M. B., & Bruno, A. C. (2019). Characterizing Complex Mineral Structures in Thin Sections of Geological Samples with a Scanning Hall Effect Microscope. Sensors, 19 (7), 1636. https://doi.org/10.3390/s19071636.

What is claimed is:

1. A method comprising:
receiving rock sample imagery of rock;
generating characterizations of the rock based at least in part on the imagery using one or more machine learning models; and
outputting the characterizations of the rock.

2. The method of claim 1, wherein the receiving, the generating and the outputting are performed by a mobile device at a field site where a rock sample is cut and imaged to generate the rock sample imagery.

3. The method of claim 2, wherein the mobile device is not in communication with a communication network that extends to a site remote from the field site.

4. The method of claim 1, wherein the generating generates confidence metrics for the characterizations of the rock.

5. The method of claim 4, wherein, responsive to one of the confidence metrics being less than a confidence metric criterion, generating a recommendation that aims to increase confidence.

6. The method of claim 5, wherein the recommendation comprises a test recommendation for performing a test on at least a portion of a rock sample at a field site.

7. The method of claim 1, wherein the outputting comprises rendering a graphical user interface to a display device.

8. The method of claim 7, wherein the graphical user interface comprises at least a portion of the rock sample imagery and one or more graphics indicative of one or more of the characteristics of the rock.

9. The method of claim 1, wherein the rock sample imagery is acquired using equipment at a field site and wherein the receiving and the generating are performed using equipment remote from the field site.

10. The method of claim 9, wherein the equipment at the field site comprises a mobile device and wherein the equipment remote from the field site comprises cloud-based equipment or a remote server.

11. The method of claim 1, comprising controlling field equipment based at least in part on the characterizations of the rock.

12. The method of claim 1, wherein the generating comprises implementing transfer learning.

13. The method of claim 12, wherein the transfer learning comprises a first domain that utilizes pre-existing rock sample imagery and a second domain that utilizes the rock sample imagery.

14. The method of claim 13, wherein the transfer learning transfers knowledge from the first set of imagery, as processed via a first one of the one or more machine learning models, to a second one of the one or more machine learning models.

15. The method of claim 1, wherein the rock sample imagery comprises imagery from a field site associated with mineral exploration.

16. The method of claim 1, wherein rock sample imagery comprises cuttings from a drill bit of a drillstring disposed in a borehole.

17. The method of claim 1, further comprising receiving data related to a rock sample, wherein the generating generates the characterizations of the rock based at least in part on the data.

18. The method of claim 17, wherein the data comprise voice recognition data generated by utterances received by a microphone of a mobile device.

19. A system comprising:
a processor;
memory operatively coupled to the processor;
a network interface; and
processor-executable instructions stored in the memory to instruct the system to:
receive rock sample imagery of rock;
generate characterizations of the rock based at least in part on the imagery using one or more machine learning models; and
output the characterizations of the rock.

20. One or more computer-readable storage media comprising computer-executable instructions executable to instruct a computing system to:
receive rock sample imagery of rock;
generate characterizations of the rock based at least in part on the imagery using one or more machine learning models; and
output the characterizations of the rock.
